(19) United States
(12) Patent Application Publication
(10) Pub. No.: US 2026/0337700 A1
(43) Pub. Date: Oct. 29, 2026

(54) METHOD AND KIT FOR DETECTING SINGLE NUCLEOTIDE POLYMORPHISMS (SNP) BY LOOP-MEDIATED ISOTHERMAL AMPLIFICATION (LAMP)

(71) Applicant: Mast Group Limited, Liverpool, Merseyside (GB)

(72) Inventors: Monika SUWARA, Liverpool, Merseyside (GB); Elizabeth Ann GILLIES, Liverpool, Merseyside (GB)

(21) Appl. No.: 18/998,803

(22) PCT Filed: Jul. 26, 2023
(86) PCT No.: PCT/GB2023/051975

§ 371 Date: Jan. 27, 2025

(30) Foreign Application Priority Data
Jul. 26, 2022 (GB) 2210921.9

(51) Int. Cl. C12Q 1/70; C12Q 1/6858

(52) U.S. Cl. CPC C12Q 1/70; C12Q 1/6858

(57) ABSTRACT

Methods for detecting and/or distinguishing one or more single nucleotide polymorphisms (SNPs) in a target nucleic acid sequence in a sample, the method including: providing a first oligonucleotide probe complimentary to a wild-type target sequence, wherein the first oligonucleotide probe includes a fluorescent label bound to an internal cytosine base and wherein said oligonucleotide probe does not have a 3′ end terminator; amplifying a target nucleic acid sequence in the sample to provide an amplified nucleic acid by a loop-mediated isothermal amplification (LAMP) in a reaction vessel with the first oligonucleotide probe and a second probe or intercalating dye and blank oligo, and probing the amplified nucleic acid sequence; detecting the presence of the target wild-type and/or a mutant nucleic acid sequence; and distinguishing between the presence of the target wild-type and/or mutant nucleic acid sequence based on the fluorescent labeling. Also provided is a kit for detecting nucleotide polymorphisms in a target nucleic acid in a sample.

### FIELD OF THE INVENTION

The present invention relates to methods and kits for detecting one or more single nucleotide polymorphisms (SNPs) using LAMP loop-mediated isothermal amplification (LAMP) and fluorescent probes.

Nucleic acid amplification is one of the most valuable tools in the life sciences fields, including application-oriented fields such as clinical medicine, in which diagnosis of infectious diseases, genetic disorders and genetic traits is particularly benefited. This process enables molecules of DNA, RNA or fragments thereof to be multiplied for further study, such as to detect target sequences.

Various methods for nucleic acid amplification exist, including polymerase chain reaction (PCR) amplification techniques, nucleic acid sequence-based amplification (NASBA), self-sustained sequence replication (3SR) and loop-mediated isothermal amplification (LAMP). PCR uses heat denaturation of double-stranded DNA products to promote the next round of DNA synthesis. 3SR and NASBA eliminate heat denaturation by using a set of transcription and reverse transcription reactions to amplify the target sequence.

The methods for nucleic acid amplification can generally amplify target nucleic acids to a similar magnitude, all with a detection limit of less than 10 copies and within an hour. They require either a precision instrument for amplification or an elaborate method for detection of the amplified products due to poor specificity of target sequence selection. Despite the simplicity and the obtainable magnitude of amplification, the requirement for a high precision thermal cycler in PCR prevents this powerful method from being widely used as a routine diagnostic tool, such as in private clinics. In contrast to PCR amplification techniques, LAMP is a method that can amplify a few copies of DNA to over 100 within an hour under isothermal conditions and with greater specificity.

In certain circumstances, there may be a need to determine the presence of mutated or otherwise altered nucleic acid sequences, which could occur in variant forms of DNA or RNA, such as genetic variants or defects including the formation of new variants of viruses and the like.

The majority of genetic variation between individuals occurs in the form of single nucleotide polymorphisms (SNPs) or, less frequently, double or triple nucleotide polymorphisms (DNPs and TNPs). Such polymorphisms can influence various characteristics of humans, animals and agricultural products, including susceptibility to diseases and pathogenic drug resistance. However, SNP mutations can also affect and/or increase the transmissibility and infectivity of pathogens, as demonstrated by the progression and continued sustainability of the SARS-CoV-2 virus in recent years.

SNPs can be detected using nucleic acid sequencing methods. However, existing non-LAMP methods can be slow, expensive and/or unreliable, often requiring specialised equipment and/or perishable assay components. For example, polymerase chain reaction (PCR) amplification techniques, which are frequently used in SNP detection, include cycling the temperature of the reaction mixture during the assay which increases its complexity and cost, and typically requires specialist equipment.

Loop-mediated isothermal amplification (LAMP) is generally quicker and does not require temperature cycling. LAMP requires 4-6 primers which can be deployed from a dry pack at room temperature, and is performed at a single continuous temperature, rather than requiring the precision temperature cycling equipment necessary for PCR. This gives it a distinct advantage over other detection methods, as it has the potential to be used in a broader range of settings. However, there are barriers to LAMP being widely adopted for the detection of SNP mutations.

For example, existing methods of SNP detection using LAMP require additional features or complicating processes, such as the incorporation of an SNP mutation at the end of a FIP/BIP LAMP primer (see, Costa-Junior et al., J. Parasit. Dis. 2021; 46 (1): 47-55). In addition, such methods require post-amplification processing steps, such as enzymatic digestion of the amplified sample with restriction enzymes (see Carvalhais at al., Front Plant Sci. 2019; 10:547) and/or a probe annealing before the SNPs can be detected (Hyman et al., 2021; bioRxiv doi: https://doi.org/10.1101/2021.03.29.437576). These additional steps increase the technical complexity, expense and time required to carry out the amplification and detection process.

Moreover, despite the additional processing, such methods commonly generate ambiguous results, which can take the form of shifts in reaction amplification times (identifiable using direct visual detection or turbidity) or changes in the baseline fluorescent signal or the fluorescence of non-specific DNA-intercalating dyes. Direct visual detection generally provides a result at the end point of a reaction, and so is unable to provide amplification data in real-time. Turbidity and non-specific intercalating dyes can provide real time data as the amplification occurs; however, this is non-specific, i.e. all amplification is detected whether this is a true positive amplification or false amplification due to mis-priming or cross specificity. Such results can be difficult to interpret if different quantities of nucleic acid templates are used in the reaction, which is, in practice, always the case with clinical samples, and it cannot be analysed in real time.

There is therefore a need to provide an improved system for the detection of SNPs that is simple to use, provides real-time results and requires minimal equipment. The improved system for the detection of SNPs may, for example, distinguish homologous sequences that differ only by the presence of one or more single nucleotide polymorphisms. Specifically, there remains a need for a SNP detection and/or distinguishing system which provides one or more of the following:
does not require post amplification manipulation (e.g., such as enzymatic digestion or probe annealing);
can distinguish between wild-type (WT) and mutant sequences unambiguously, optionally in an unbiased manner;
does not require that amplification times or baseline fluorescence levels be compared or that complex statistical analysis be used in order to obtain a result;
does not require replicative testing in order to obtain an unbiased, unambiguous result; and/or
is suitable for use in the clinic wherein samples arrive at different concentrations.

In accordance with a first aspect of the invention, there is provided a method for detecting single nucleotide polymorphisms (SNPs) in a target nucleic acid sequence in a sample, the method comprising: a) providing a first oligonucleotide probe complementary to a wild-type target sequence, wherein the first oligonucleotide probe comprises a fluorophore label bound to an internal cytosine base and wherein said oligonucleotide probe does not have a 3′ end terminator; b) amplifying a target nucleic acid sequence in the sample to provide an amplified nucleic acid by a loop-mediated isothermal amplification in a reaction vessel with the first oligonucleotide probe and a second probe or intercalating dye, and probing the amplified nucleic acid sequence; c) detecting the presence of the target wild-type and/or a mutant nucleic acid sequence; and d) distinguishing between the presence of the target wild-type and/or mutant nucleic acid sequence based on the fluorescent labeling. In another arrangement the first oligonucleotide probe is complementary to a mutant sequence of a wild-type target sequence.

Since the fluorescent probe is present during the amplification process, the fluorescent probe binds to the target sequence and can be incorporated into the amplicon by BST DNA polymerase. The fluorescence of the attached fluorescent label allows the presence of the amplified target nucleic acid sequence to be unambiguously detected in real-time during the reaction, in the reaction vessel itself. As fluorescence is detectable during the reaction, it is particularly advantageous because no post-reaction manipulation of the amplified nucleic acid is required. As such, a simpler and more efficient detection of target nucleic acid sequences may be achieved.

In addition, use of the second probe or intercalating dye enables the real-time detection of a further amplified target nucleic acid sequence, and/or amplified nucleic acid in the reaction as a whole.

Using the first and second labelled probes in the same LAMP reaction enables multiple sequences, including single or multiple SNPs or a wild-type sequence and corresponding mutant sequence, to be detected and distinguished using a single assay. The use of the competing second probe (mutant probe) and the first probe (wild type probe) in the same reaction also increases the specificity of detection, because the first and second probes compete for binding sites during the reaction. The presence of additional probes in the reaction reduces the chance of a probe binding to a nucleic acid sequence which is not fully complementary, and producing a false positive result. In the absence of a competing probe (second probe), the first probe (wild type probe) is more likely to bind non-specifically to the mutant sequence which differs from the wild type sequence by a single nucleotide. A competing probe having a complete sequence match to the mutant sequence will bind to the sequence having a point mutation (SNP) with higher affinity than the wild type probe. The competing mutant probe will block the binding site in the mutant sequence, preventing the wild type probe from non-specific annealing and generating false amplification signal in the presence of the mutant sequence. The oligonucleotide probes according to the invention do not have a 3′ end terminator. The 3′ end terminator that is not present may be ddNTP, for example. The absence of a 3′ end terminator enables incorporation of the labelled oligonucleotide into the amplicon. Thus, the 3′ end of the probe is not “blocked”.

In one aspect of the invention, the first probe is complementary to a target nucleic acid sequence. Any suitable target sequence may be used. The target sequence may be selected from a wild-type target sequence of any organism. In one arrangement, the first probe may be a wild-type DNA or RNA sequence, optionally having a single point mutation positioned opposite the nucleotide located at or near the 3′ end of the probe. This arrangement is particularly advantageous when a competing probe is not present, e.g. a second probe that is complementary to a mutant sequence of the wild-type target sequence, or a competing sequence (e.g. a so-called “blank oligo”) is not present, as it reduces potential costs and assay complexity.

In one arrangement, the target nucleic acid sequence may be a virus. Any suitable virus may be used. For example, the virus may be selected from SARS-CoV-2 or a variant thereof.

In an aspect of the invention, the second probe may be selected from a probe complementary to a mutant sequence of the wild-type target sequence, or a generic probe, such as a generic probe capable of detecting both the wild-type target sequence and the mutant sequence of the wild-type target sequence. In one aspect, the second probe may be selected from a probe complementary to a mutant sequence of the wild-type target sequence. In an alternative aspect, the second probe may be selected from a generic probe capable of detecting both the wild-type target sequence and the mutant sequence of the wild-type target sequence. In another arrangement, the second probe may be selected from a probe complementary to a wild-type target sequence. This arrangement is suitable when the first probe is selected from a probe complementary to a mutant sequence of a wild-type target sequence.

In one arrangement, the method of clause 1, step b), for example, involves providing the intercalating dye or the generic probe. The generic probe may be similar to the first probe, except that it targets a nucleic acid sequence that is common for both wild-type target sequence and the mutant sequence. In yet a further arrangement, the generic probe may not comprise the nucleotide at which the SNP occurs. In yet still a further arrangement, there is no overlap between the sequences of the first probe and the generic probe. In still yet a further arrangement, the generic probe targets a different gene in a sample to the first and/or second probes wherein the different target gene has a more conserved sequence than the sequence targeted than the wild type and/or mutant probes, optionally wherein an additional set of primers is be used. Such generic probes allow detection of both wild type and mutant sequence amplification, which enables comparison of the of the wild type and/or mutant sequence amplification to the total amplification during the LAMP reaction.

In one or more embodiments of the invention at least one of the probes comprises a single- or double-point mutation. Optionally, each of the probes comprises a single- or double-point mutation. Probes comprising single- or double-point mutations enable the detection of corresponding single- or double point-mutations in a sample nucleic acid sequence.

In some embodiments of the invention a second probe which is complementary to a mutant sequence of the wild-type target sequence is used, wherein the second probe further comprises a fluorescent label bound to an internal cytosine base and does not have a 3′ end terminator; and wherein at least one of the first and second probes contains a single- or double-point mutation at or near the 3′ end.

In some aspects of the invention, one or more so-called “blank oligos” may be used. Any suitable blank oligo may be used. The blank oligo is absent a fluorophore label, optionally the blank oligo may comprise the same sequence as the first probe. For example, the blank oligo may comprise one or more SNP mutations at or near the 3′ end or in the middle of the sequence. In one arrangement, a SNP at the 3′ end may include a SNP at the terminal nucleotide base at the 3′ end of a nucleic acid sequence, and a SNP near the 3′ end may include a SNP at the nucleotide base second from the 3′ end. In another arrangement, the one or more blank oligos may be the same or different.

In some aspects of the invention, a blank oligo may be complementary to a wild-type target sequence. In this arrangement, a first probe may comprise one or more SNP mutations at or near the 3′ end or in the middle of a sequence. The first probe may be complementary to a mutant sequence of the wild-type sequence. In another arrangement, one or more probes may comprise one or more SNP mutations at or near the 3′ end or in the middle of a sequence.

The one or more probes may be complementary to one or more mutant sequences of a wild-type.

The use of a blank oligo is intended to increase the specificity of the reaction by competing for binding sites with the fluorescent probe so that the oligonucleotide which more closely mirrors the target sequence can be incorporated into the amplified nucleic acid, without requiring the presence of a second probe. A blank oligo may be used when it is not possible to design or manufacture a functional competing second probe labelled with a fluorophore distinguishable from the first probe or if the mutant contains several SNPs that cannot be covered by a single probe, but they are located in the same position or close proximity to each other and they all generate the same phenotype (for example antibiotic resistance). Further, multiple different blank oligos may be used in the same reaction if a mutant sequence comprises several different point mutations close to one another that cannot be covered by 1 or 2 fluorescent probes. In one arrangement, the second probe may be selected from a generic probe, and the method involves providing the intercalating dye or the generic probe, and one or more blank oligos. In a further arrangement, the one or more blank oligos may comprise one or more SNP mutations at or near 3′ end or one or more SNPs in the middle of the sequence. For example, in some embodiments, the method comprises providing the intercalating dye or the generic probe, and one or more blank oligos having the same sequence as the first probe but absent a fluorescent label, and comprising one or more SNP mutations at or near 3′ end.

In an aspect of the invention, the blank oligo may be provided at an equimolar concentration to a first probe. In some arrangements, the blank oligo may be provided in excess concentration to a first probe. If the blank oligo is provided at an excess concentration in the absence a second probe that competes with the first probe, the reaction specificity may be further increased. The reaction specificity may be even further increased by the excess blank oligo increasing competition for access to the target binding site, especially when the blank oligo's target sequence is present in a sample. By way of example, when there is a first probe complementary to a wild-type target sequence but no competing second probe and a blank oligo in excess complementary to a mutant sequence of the wild-type target sequence, there will be a weak or absent fluorescent signal from incorporation of the first probe into growing amplicons when only the mutant sequence of the wild-type is present. In the same example, when only the wild-type sequence is present in a sample, the first fluorescent probe will generate a signal. When samples are detected by a real-time detection LAMP assay, the difference in CT values between samples comprising wild-type sequences and samples comprising mutant sequences of the wild-type can be more distinguished when using a blank oligo in excess with a first probe in the absence of a competing second probe. In some embodiments, the method comprises providing the intercalating dye or the generic probe, and one or more blank oligos, wherein at least one blank oligo has the same sequence as the first probe but is absent a fluorescent label, and comprises one or more SNPs in the middle of the sequence. In an alternative arrangement, the method comprises providing the intercalating dye or the generic probe, and one or more blank oligos absent a fluorescent label, wherein at least one blank oligo has the same sequence as a wild-type sequence and the first probe comprises a mutant sequence of the wild-type target sequence.

In some aspects of the invention, the method may be carried out in the presence of a buffer. Any suitable buffer may be used. In one arrangement, the buffer may be a Tris buffer, which is a buffer that may be used in a broad range of LAMP reactions and nucleic acid samples. In another arrangement, when the one or more blank oligos comprises one or more SNPs in the middle of the sequence, the buffer may be a CHES CAPSO buffer. The use of a buffer, specifically a CHES CAPSO buffer, in the method allows probes to be designed where the single- or double-point mutation is in the middle of the probe sequence, which is advantageous where designing a probe with the mutation at the 3′ end is not possible. Tris buffer is compatible with most LAMP assays, however CHES CAPSO buffer may provide improved specificity for problematic primers that are prone to generate primer dimers.

In some embodiments where the method omits a blank oligo, the method may comprise providing the intercalating dye or the generic probe wherein the first probe is specific to wild-type DNA or RNA sequence having a single point mutation positioned opposite the nucleotide located at or near the 3′ end of the probe.

Any suitable fluorescent label or fluorophore label may be used in the invention. In some aspects, the fluorescent label, the fluorophore label, and/or the intercalating dye may comprise one or more of the following: TAMRA, FAM, Cy5, SYBR Green, EvaGreen, JOE, TET, HEX, ROX, ALEXA, ATTO and V13, or any other dye suitable for nucleic acid detection. In one arrangement, the fluorescent label, the fluorophore label, or the intercalating dye may be different, which allows a distinction to be drawn in relation to the detection. For example, the first and second probes may comprise different labels, or the first probe and the intercalating dye may comprise different labels.

In some aspects of the invention, the first probe is configured to function as a loop-mediated isothermal amplification primer. In another aspect of the invention, the second probe is configured to function as a loop-mediated isothermal amplification primer. In another aspect of the invention, the one or more blank oligos are configured to function as a loop-mediated isothermal amplification primer. In a further aspect of the invention, the first and/or second probe is configured to function as a loop-mediated isothermal amplification primer. In a further aspect of the invention, the first and/or second probe and/or one or more blank oligo(s) is configured to function as a loop-mediated isothermal amplification primer. In some aspects of the invention, the loop-mediated isothermal amplification primer is an LF or LB loop primer.

In another aspect of the invention, the first probe, second probe, and/or target nucleic acid may be a DNA sequence or an RNA sequence. In a further aspect of the invention, the first probe, second probe, blank oligo and/or target nucleic acid may be a DNA sequence or an RNA sequence. Any suitable DNA sequence or RNA sequence could be used. In one aspect of the invention, the first probe may be a DNA sequence or an RNA sequence. In a further aspect of the invention, the second probe may be a DNA sequence or an RNA sequence. In yet another aspect of the invention, the first probe may be a DNA sequence or an RNA sequence and the second probe may be a DNA sequence or an RNA sequence. In some aspects of the invention, each of the probes, primers and/or blank oligos used in the LAMP reaction may be DNA sequences. In some other aspects of the invention, all of the probes, primers and blank oligos used in the LAMP reaction may be DNA sequences.

Optionally, the loop-mediated isothermal amplification may be carried out using an RB (tris) buffer.

In some embodiments, the first or second probe, or blank oligo may comprise one or more of the following sequences:

SEQ ID NO 1:
5′ GTATGGTTGGTAACCAACA/IFAMdCN/CATT 3′
(SARS-CoV-2 WT probe
S gene for distinction of A23063T
N501Y point mutation)
SEQ ID NO. 2:
5′ AAAGGAAAGTAACAATTAAAAC/16-TAMN-dC/TTC 3′
(SARS-CoV-2 WT probe S gene for distinction
of E484K point mutation)
SEQ ID NO. 3:
5′ AAAGGAAAGTAACAATTAAAACCTTT 3′
(SARS-CoV-2 mutant blank oligo
for distinction of E484K point mutation).

In some aspects of the invention, the presence of the target nucleic acid and/or mutant nucleic acid sequence can be detected by loop-mediated isothermal amplification (LAMP). This may be achieved with or without the need for any post-amplification manipulation, preferably without the need for any post-amplification manipulation. This absence of post-amplification manipulation is particularly advantageous because it reduces the complexity of the method, and it allows for quicker detection of an SNP in the sample.

In some embodiments, methods of the invention enable the detection of an SNP, wherein the target nucleic acid is from a virus, such as SARS-CoV-2 or a variant thereof.

The LAMP process may use any suitable primer set. A suitable primer set comprises at least FIP, BIP, F3 and B3 primers, and may optionally comprise loop F and/or loop B primers. In some arrangements, loop F and/or loop B primers may be used to increase the speed and/or the sensitivity of the LAMP reaction. In some arrangements, only the FIP, BIP, F3 and B3 primers may be used, e.g., where is it not possible to design a primer set comprising loop F and/or loop B primers, and/or to reduce the chance of false amplification.

In some aspects of the invention, the LAMP reaction may be carried out in any suitable vessel. In further aspects of the invention, the detection of amplified nucleic acid sequences may be carried out in any suitable vessel. In yet further aspects of the invention, the LAMP reaction and the detection of amplified nucleic acid sequences may both be carried out in the same reaction vessel. If the LAMP reaction and the detection of amplified nucleic acid sequences is carried out in the same reaction vessel, then the reaction can be analysed in real-time, using the equipment necessary to carry out the method.

In an alternative aspect of the invention, there is provided a kit for detecting nucleotide polymorphisms in a target nucleic acid sequence in a sample. The kit comprises: a) a first oligonucleotide probe complementary to a wild-type target sequence, wherein the first oligonucleotide probe comprises a fluorescent label bound to an internal cytosine base and wherein said oligonucleotide probe sequence does not have a 3′ end terminator; and b) a loop-mediated isothermal amplification reagent buffer, enzyme, dNTPs and loop-mediated isothermal amplification primers; wherein the first probe is configured to be used in a single reaction vessel.

In some aspects of the invention, the kit may further comprise an intercalating dye or a second oligonucleotide probe, wherein the second oligonucleotide probe is selected from a probe complementary to a mutant sequence of the wild-type target sequence, or a generic probe capable of detecting both the wild type and mutant target sequence; wherein the second oligonucleotide probe and/or intercalating dye comprise a further fluorescent label distinguishable from the first probe; and the first probe, the second probe and/or the intercalating dye are configured to be used in a single reaction vessel. In some aspects the kit may further comprise one or more blank oligos. The kit may further comprise one or all of a loop-mediated isothermal amplification reagent buffer, enzyme, deoxyribonucleoside 5′-triphosphates (dNTPs) and loop-mediated isothermal amplification primers. Optionally, the kit may comprise an RB (tris) buffer.

In an alternative arrangement of the kit, there is provided a) a first oligonucleotide probe complementary to a mutant sequence of a wild-type, wherein the first oligonucleotide probe comprises a fluorescent label bound to an internal cytosine base and wherein said oligonucleotide probe sequence does not have a 3′ end terminator; and b) a loop-mediated isothermal amplification reagent buffer, enzyme, dNTPs and loop-mediated isothermal amplification primers; wherein the first probe is configured to be used in a single reaction vessel, the kit may further comprise an intercalating dye or a second oligonucleotide probe, wherein the second oligonucleotide probe is selected from a probe complementary to a wild-type sequence, or a generic probe capable of detecting both the wild type and mutant target sequence; wherein the second oligonucleotide probe and/or intercalating dye comprise a further fluorescent label distinguishable from the first probe; and the first probe, the second probe and/or the intercalating dye are configured to be used in a single reaction vessel. In some aspects the kit may further comprise one or more blank oligos. The one or more blank oligos may be complementary to a wild-type sequence.

Optionally, the kit may comprise additional labelled probes. For example, the kit may provide 2 or at least 2 labelled probes, 3 or at least 3 labelled probes, 4 or at least 4 labelled probes, 5 or at least 5 labelled probes, 6 or at least 6 labelled probes, or 10 or at least 10 labelled probes. In another arrangement, the kit may provide between 2-10 labelled probes, between 2-9 labelled probes, between 2-8 labelled probes, between 3-7 labelled probes, or between 3-6 labelled probes. In one arrangement, any suitable number of labelled probes may be used. Such probes may be used to detect additional SNPs or wild-type sequences.

In some aspects of the invention, the kit may comprise at least one probe comprising a mutation. For example, at least one probe may comprise a single- or double-point mutation, optionally at or near the 3′ end.

In some further aspects of the invention, the kit may comprise a second probe that is complementary to a mutant sequence of the wild-type target sequence. In one arrangement, the second probe may further comprise a fluorophore label. This label may be bound to an internal cytosine base, and optionally the second probe does not have a 3′ end terminator. In a further arrangement, at least one of the first and/or second probes comprises a single- or double-point mutation at or near the 3′ end. In another arrangement, the kit may comprise a second probe that is complementary to a wild-type target sequence. This arrangement is suitable when the first probe is complementary to a mutant sequence of the wild-type target sequence.

In some aspects of the invention, the kit may comprise a second probe that is complementary to a mutant sequence of the wild-type target sequence. In one arrangement, the second probe may comprise a fluorophore label bound to an internal cytosine base and does not have a 3′ end terminator. Optionally, at least one of the first and second probes may contain a single- or double-point mutation at or near the 3′ end.

In a further aspect of the invention, the kit provides a second probe that may be selected from a probe complementary to a mutant sequence of the wild-type target sequence, or a generic probe capable of detecting both the wild-type target sequence and the mutant sequence of the wild-type target sequence. In one aspect, the second probe may be selected from a probe complementary to a mutant sequence of the wild-type target sequence. In an alternative aspect, the second probe may be selected from a generic probe capable of detecting both the wild-type target sequence and the mutant sequence of the wild-type target sequence.

Where the kit provides an intercalating dye or a generic probe, the kit may further provide one or more so-called “blank oligos” that may be used. Any suitable blank oligo may be used. The blank oligo is absent a fluorophore label. Optionally the blank oligo may comprise the same sequence as the first probe and may comprise one or more SNP mutations at or near the 3′ end or in the middle of the sequence. In one arrangement, a SNP at the 3′ end includes a SNP at the terminal nucleotide base at the 3′ end of a nucleic acid sequence, and a SNP near the 3′ end includes a SNP at the nucleotide base second from the 3′ end. Where more than one blank oligos are used, the one or more blank oligos may be the same or different. In other arrangements, the blank oligo may comprise a wild-type sequence and a first probe may comprise a mutant sequence of the wild-type comprising one or more SNP mutations at or near the 3′ end or in the middle of the sequence.

Alternatively, or in addition, where the kit comprises an intercalating dye or the generic probe, and one or more blank oligos, the one or more blank oligos may have the same sequence as the first probe but absent a fluorophore label. In a further arrangement, the one or more blank oligos may comprise one or more SNP mutations at or near 3′ end or one or more SNPs in the middle of the sequence. In yet another arrangement, multiple different blank oligos may be used in the same reaction if a mutant sequence comprises several different point mutations close to one another that cannot be covered by 1 or 2 fluorescent probes.

In some aspects of the invention, the kit may comprise a buffer. Any suitable buffer may be used. In one arrangement, the buffer may be a RB (tris) buffer, which is a buffer that may be used in a broad range of LAMP reactions and nucleic acid samples. In another arrangement, when the one or more blank oligos comprises one or more SNPs in the middle of the sequence, the buffer may be a CHES CAPSO buffer. The use of a buffer, specifically a CHES CAPSO buffer, in the method allows probes to be designed where the single- or double-point mutation is in the middle of the probe sequence, which is advantageous where designing a probe with the mutation at the 3′ end is not possible. In some arrangements, the second probe may be selected from a generic probe, and the kit provides the intercalating dye or the generic probe. In some arrangements, the first probe may comprise one or more SNPs in the middle of the sequence, the one or more blank oligos be complementary to a wild-type target sequence, and the buffer may be CHES CAPSO buffer.

In some embodiments, the kit omits a blank oligo, and the first probe is specific to wild-type DNA or RNA sequence having a single point mutation positioned opposite the nucleotide located at or near the 3′ end of the probe.

Any suitable fluorescent label or fluorophore label may be used with probes in the kit. The fluorescent label, the fluorophore label, or the intercalating dye may comprise one or more of the following: TAMRA, FAM, Cy5, SYBR Green, EvaGreen, JOE, TET, HEX, ROX, ALEXA, ATTO and V13, or any other dye suitable for nucleic acid detection. In one arrangement, the fluorescent label, the fluorophore label, or the intercalating dye that may be different. For example, the first and second probes may comprise different labels, or the first probe and the intercalating dye may comprise different labels.

In some aspects of the invention, a kit may be provided wherein the first probe may be configured to function as a loop-mediated isothermal amplification primer. In other aspects of the invention, the second probe is configured to function as a loop-mediated isothermal amplification primer. In other aspects of the invention, the blank oligo is configured to function as a loop-mediated isothermal amplification primer. In a further aspects of the invention, the first and/or second probe is configured to function as a loop-mediated isothermal amplification primer. In a further aspect of the invention, the first probe, second probe, and/or blank oligo is configured to function as a loop-mediated isothermal amplification primer. In some aspects of the invention, the isothermal amplification primer is a LF or LB loop primer.

In addition or alternatively, the first probe, second probe, and/or target nucleic acid may be a DNA sequence or an RNA sequence. In addition or alternatively, the first probe, second probe, blank oligo, and/or target nucleic acid may be a DNA sequence or an RNA sequence. Any suitable DNA sequence or RNA sequence could be used. In one aspect of the invention, the first probe may be a DNA sequence or an RNA sequence. In a further aspect of the invention, the second probe may be a DNA sequence or an RNA sequence. In yet another aspect of the invention, the first probe may be a DNA sequence or an RNA sequence and the second probe or blank oligo may be a DNA sequence or an RNA sequence. In some aspects of the invention, each of the probes, primers and/or blank oligos may be DNA sequences. In some other aspects of the invention, all of the probes, primers and blank oligos may be DNA sequences.

In a preferred embodiment, the first or second probe or blank oligo comprises one or more of the following sequences:

SEQ ID NO. 1:
5′ GTATGGTTGGTAACCAACA/IFAMdCN/CATT 3′
(SARS-CoV-2 WT probe S gene for distinction
of A23063T N501Y point mutation);
SEQ ID NO. 2:
5′ AAAGGAAAGTAACAATTAAAAC/16-TAMN-dC/TTC 3′
(SARS-CoV-2 WT probe S gene for distinction
of E484K point mutation);
and
SEQ ID NO. 3:
5′ AAAGGAAAGTAACAATTAAAACCTTT 3′
(SARS-CoV-2 mutant blank oligo
for distinction of E484K point mutation).

The kit may be configured to detect the presence of the target nucleic acid and/or mutant nucleic acid sequences which can be detected by loop-mediated isothermal amplification (LAMP). In some aspects of the invention, the kit may be configured to allow detection of the presence of the target wild-type and/or mutant nucleic acid sequences during LAMP without the need for any post-amplification manipulation. Preferably the kit may be suitable for detecting such nucleic acid sequences without the need for any post-amplification manipulation.

In some embodiments, the kit may be suitable for detecting a target nucleic acid from a virus, such as the virus SARS-CoV-2 or a variant thereof.

The kit may be configured for use in a LAMP process wherein the LAMP may use any suitable primer set. A suitable primer set comprises at least FIP, BIP, F3 and B3 primers, and may optionally comprise loop F and/or Loop B primers.

### BRIEF DESCRIPTION OF THE DRAWINGS

FIGS. 1a-1b are amplification plots for LAMP assays comprising two fluorescent probes according to Example 1.

FIGS. 2a-2c are amplification plots for LAMP assays comprising a single fluorescent probe and an intercalating dye according to Example 2.

FIGS. 3a-3d are amplification plots for LAMP assays comprising a single fluorescent probe, a blank oligo and an intercalating dye, wherein the SNP on the blank oligo is positioned at the 3′ end according to Example 2a.

FIGS. 4a-4d are amplification plots for LAMP assays comprising a single fluorescent probe, a blank oligo and an intercalating dye, wherein the SNP on the blank oligo is positioned at the nucleotide second from the 3′ end according to Example 2b.

FIGS. 5a-5d are amplification plots for LAMP assays comprising a single fluorescent probe, a blank oligo and an intercalating dye, wherein the blank oligo comprises a double point mutation positioned at the nucleotides at the 3′ end and second from the 3′ end according to Example 2c.

FIGS. 6a-6c are amplification plots for LAMP assays which were control experiments for the experiments of FIGS. 3a-3d, FIGS. 4a-4d and 5a-5d performed in the absence of blank oligo according to Example 2d.

FIG. 7a-7d are amplification plots for LAMP assays comprising a single fluorescent probe, a blank oligo and an intercalating dye, wherein the SNP on the blank oligo is positioned in the middle of the blank oligo sequence, and carried out under RB (tris) buffer conditions according to Example 3a.

FIG. 8a-8d are amplification plots for LAMP assays comprising a single fluorescent probe, a blank oligo and an intercalating dye, wherein the SNP on the blank oligo is positioned at in the middle of the blank oligo sequence, and carried out under RB (Tris) buffer conditions according to Example 3b.

FIGS. 9a-9d are amplification plots for LAMP assays comprising a single fluorescent probe, a blank oligo and an intercalating dye, wherein the SNP on the blank oligo is positioned at in the middle of the blank oligo sequence, and carried out under CHES CAPSO buffer conditions according to Example 3c.

FIGS. 10a-10d are amplification plots for LAMP assays comprising a single fluorescent probe, a blank oligo and an intercalating dye, wherein the SNP on the blank oligo is positioned at in the middle of the blank oligo sequence, and carried out under CHES CAPSO buffer conditions according to Example 3d.

FIGS. 11a-11d are amplification plots for LAMP assay or SARS-CoV-2 RNA comprising fluorescent wild type probe and a generic control probe according to Example 4.

### DETAILED DESCRIPTION

According to one aspect of the invention, a method and kit is provided which is configured to detect SNPs using two fluorescently labelled oligonucleotide probes in the same LAMP reaction. The first probe is an oligonucleotide comprising a fluorophore label bound to an internal cytosine base, and which does not have a 3′ end terminator. The first probe is complementary to a wild-type target sequence. The second probe is an oligonucleotide sequence comprising a fluorophore label, which is distinguishable from the fluorescent label of the first probe, bound to an internal cytosine base and which does not have a 3′ end terminator. The second probe is complementary to a mutant sequence of the wild-type sequence, and comprises a single- or double-point mutation at or near the 3′ end of the oligonucleotide sequence. In some embodiments, at or near the 3′ end comprises the terminal nucleotide base at the 3′ end, and the nucleotide base second from the 3′ end.

The target nucleic acid sequence in the sample is amplified using LAMP to provide an amplified nucleic acid in a reaction vessel with the oligonucleotide first probe, and probing the amplified nucleic acid sequence. The target nucleic acid sequence is amplified using a LAMP reaction in the presence of both the first and the second probes. In some embodiments the LAMP reaction is a single-vessel reaction. The two probes compete for binding sites on the amplified nucleic acid sequence. Thus, an amplified nucleic acid sequence comprising the wild-type sequence complementary to the first probe can be detected by detecting the fluorescence of the fluorescent label of the first probe, an amplified nucleic acid sequence comprising the mutant sequence complementary to the second probe can be detected by detecting the fluorescence of the fluorescent label of the second probe. The presence of the target wild-type and/or mutant nucleic acid sequences can, therefore, be detected using their fluorescence, and can be distinguished based on their fluorescent labelling.

According to a further aspect of the invention, there is provided a method for detecting SNPs using a fluorescently labelled oligonucleotide probe and a blank oligo in the same LAMP reaction. The fluorescently labelled oligonucleotide probe is an oligonucleotide probe that is complementary to a WT target sequence (WT fluorescent probe). The blank oligo is an oligonucleotide sequence which has the same sequence as the WT fluorescent probe, but it is not labelled with a fluorophore and it contains one or more SNP mutation(s) (i.e., single- or double-point mutation(s)) at or near the 3′ end of the oligonucleotide sequence. In some embodiments, at or near the 3′ end comprises the terminal nucleotide base at the 3′ end, and the nucleotide base second from the 3′ end.

The target nucleic acid sequence in the sample is amplified using a LAMP reaction to provide an amplified nucleic acid in a reaction vessel, in the presence of first probe and the blank oligo, wherein the amplified nucleic acid sequence is probed. The one or more fluorescence signals are used to detect the presence of the amplified WT sequence and, if the intercalating dye or generic probe are present, the presence of either the WT or SNP sequence can be distinguished.

According to a further aspect of the invention, there is provided a method for detecting SNPs using a fluorescently labelled oligonucleotide probe and a blank oligo in the same LAMP reaction. The fluorescently labelled oligonucleotide probe is an oligonucleotide probe which has the same sequence as the WT target sequence, but it contains one or more SNP mutation(s) (i.e., single- or double-point mutation(s)) at or near the 3′ end of the oligonucleotide sequence. In some embodiments a mutation at or near the 3′ end of the oligonucleotide sequence may include a SNP at a terminal nucleotide base located at the 3′ end, and/or a SNP at a nucleotide base second from the 3′ end. The blank oligo is an oligonucleotide sequence which has the same sequence as the target WT sequence, but it is not labelled with a fluorophore.

According to further aspect of the invention, there is provided a method for detecting SNPs using a fluorescently labelled oligonucleotide probe and a blank oligo in the same LAMP reaction, wherein the SNP(s) are located substantially in the middle portion of the blank oligo. The fluorescently labelled oligonucleotide probe is an oligonucleotide probe comprising a fluorophore label bound to an internal cytosine base, and which does not have a 3′ end terminator. The first probe is complementary to a WT target sequence. The blank oligo is an oligonucleotide which has the same sequence as the WT fluorescent probe, but it is not labelled with a fluorophore and it contains one or more SNP mutation(s) (i.e., single- or double-point mutation(s)) in the middle of the blank oligo sequence. In some embodiments, the middle of the sequence comprises any nucleotide between the second nucleotide from the 3′ end and the 5′ end or the sequence.

According to further aspect of the invention, there is provided a method for detecting SNPs using a fluorescently labelled oligonucleotide probe and a blank oligo in the same LAMP reaction, wherein the SNP(s) are located substantially in the middle portion of the fluorescently labelled probe. The fluorescently labelled oligonucleotide probe is an oligonucleotide probe comprising a fluorophore label bound to an internal cytosine base, and which does not have a 3′ end terminator. The blank oligo is complementary to a WT target sequence and is not labelled with a fluorophore. The fluorescently labelled probe has the same sequence as the blank oligo, but it contains one or more SNP mutation(s) (i.e., single- or double-point mutation(s)) in the middle of the oligonucleotide probe sequence. In some embodiments, a mutation the middle of the oligonucleotide probe sequence may include one or more SNPs in any nucleotide between the second nucleotide from the 3′ end and the 5′ end of the sequence. The reaction may further comprise a fluorescent intercalating dye or a generic fluorescent probe, the fluorescence of which is distinguishable from the first probe. A generic fluorescent probe targets a nucleic acid sequence that is common for both WT and mutant (SNP) samples, but does not comprise the nucleotide at which the SNP occurs. The fluorescent intercalating dye or a generic probe can provide a detectable fluorescence control signal in the presence of, and corresponding to, both the WT and mutant target sequences in a sample. In some arrangements, the generic probe may be designed to target a different target gene than the first probe, wherein the different target gene has a more conserved sequence than the sequence targeted than the wild type and mutant variants. In some arrangements where the generic probe targets a different target gene, a further set of primers may be used. In some arrangements there is no overlap between the sequences of the first probe and the generic probe.

The target nucleic acid sequence in the sample is amplified using a LAMP reaction to provide an amplified nucleic acid in a reaction vessel, in the presence of first probe and the blank oligo, wherein the amplified nucleic acid sequence is probed. The one or more fluorescence signals are used to detect the presence of the amplified WT sequence and, if the intercalating dye or generic probe are present, the presence of either the WT or SNP sequence can be distinguished.

According to a further aspect of the invention, the one or more blank oligos may be provided at a concentration that is equal to a concentration of a first fluorescently labelled probe. In another arrangement, the concentration of the one or more blank oligos exceeds the concentration of the first probe. In some arrangements, the concentration of the one or more blank oligos is provided at about 1.5 times or more, about 2 times or more, or about 4 times or more, greater than the concentration of the first probe. In some arrangements, the concentration of the one or more blank oligos is provided at from about 1.25 to about 6 times, from about 1.5 to about 5 times, from about 1.75 to about 4.75 times, from about 1.5 to about 4.5 times, from about 1.75 to about 4.0 times, from about 2 to about 3.75 times, from about 2.25 to about 3.5 times, from about 2.5 to about 3.25 times, or from 2.75 to about 3 times, greater than the concentration of the first probe. In some arrangements, the concentration of the one or more blank oligos is about 2 times or about 4 times greater than the concentration of the first probe.

In a LAMP reaction comprising the first probe and a blank oligo and where a second probe that competes with the first probe is absent, it is particularly advantageous that the blank oligo is provided at an excess concentration because the reaction specificity can be further increased. The reaction specificity can be further increased by the excess blank oligo increasing competition for access to the target binding site, especially when the blank oligo's target sequence is present in a sample. By way of example, when there is a first probe complementary to a wild-type target sequence but no competing second probe and a blank oligo in excess complementary to a mutant sequence of the wild-type target sequence, there will be a weak or absent fluorescent signal from incorporation of the first probe into growing amplicons when only the mutant sequence of the wild-type is present. In the same example, when only the wild-type sequence is present in a sample, the first fluorescent probe will generate a signal. When samples are detected by a real-time detection LAMP assay, the difference in CT values between samples comprising only wild-type sequences and samples comprising only mutant sequences of the wild-type can be more distinguished when using a blank oligo in excess with a first probe in the absence of a competing second probe.

According to a further embodiment of the invention, there is provided a method for detecting SNPs using a fluorescently labelled oligonucleotide probe in a LAMP reaction, wherein the method omits a blank oligo, and the first probe is specific to a WT nucleic acid sequence having a single point mutation positioned opposite the nucleotide located at or near the 3′ end of the probe.

The fluorescently labelled oligonucleotide probe is an oligonucleotide probe comprising a fluorophore label bound to an internal cytosine base, and which does not have a 3′ end terminator. The first probe is complementary to a WT target sequence.

The reaction may further comprise a fluorescent intercalating dye or a generic fluorescent probe, the fluorescence of which is distinguishable from the first probe. The fluorescent intercalating dye or a generic probe can provide a detectable fluorescence control signal in the presence of both the WT and mutant target sequences in a sample.

The target nucleic acid sequence in the sample is amplified using a LAMP reaction to provide an amplified nucleic acid in a reaction vessel, in the presence of the first probe, wherein the amplified nucleic acid sequence is probed. The one or more fluorescence signals are used to detect the presence of the amplified WT sequence and, if the intercalating dye or generic probe are present, the presence of either the WT or SNP sequence can be distinguished.

This method is simplified and so easier and less expensive to carry out, as it does not require a competing probe or blank oligo.

In some embodiments, the LAMP reaction and the detection of amplified nucleic acid sequences are carried out in the same vessel.

In some embodiments, the step of providing an intercalating dye or a second probe wherein the second probe (selected from a probe complementary to a mutant sequence of the wild-type target sequence, or generic probe capable of detecting both the wild-type and mutant target sequence) is not included in the method of the invention.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

The term “nucleic acid” refers to a nucleotide polymer unless stated otherwise. Nucleic acids include RNA, DNA, or chimeric DNA-RNA polymers or oligonucleotides, and analogs thereof.

The term “nucleotide,” as used herein refers to a subunit of a nucleic acid consisting of a phosphate group, a 5-carbon sugar and a nitrogenous base. The 5-carbon sugar found in RNA is ribose. In DNA, the 5-carbon sugar is 2′-deoxyribose.

The term “target sequence” as used herein, refers to the particular nucleotide sequence of a target nucleic acid that is to be detected. The “target sequence” includes the complexing sequences to which oligonucleotides complex during a detection process. The target sequence may form part of a larger nucleic acid sequence.

The interchangeable terms “oligomer,” “oligo,” and “oligonucleotide” refer to a nucleic acid having generally less than 1,000 nucleotide residues, including nucleic acids in a range of about 5, 9, 10, 15, 20, 25, 30, 35, 40, 45, 50, 55 and/or 60 nucleotide residues and about 200 or 500 to about 900 residues.

The term “probe” refers to a nucleic acid oligonucleotide that can hybridise specifically to a target sequence in a nucleic acid, or in an amplified nucleic acid, under conditions that promote hybridization to allow detection of the target sequence or amplified nucleic acid.

The term “blank oligo” refers a nucleic acid oligonucleotide that can hybridise specifically to a target sequence in a nucleic acid, or in an amplified nucleic acid, under conditions that promote hybridization, but does not allow detection of the target sequence or amplified nucleic acid.

The interchangeable terms “label” and “fluorescent label”, “fluorophore” and “fluorophore label” refer to a moiety or compound that is detected or leads to a detectable signal.

The term “amplification” refers to any known procedure for obtaining multiple copies of a target nucleic acid sequence or its complement or fragments thereof. The multiple copies may be referred to as amplicons or amplification products.

The term “amplicon,” which is used interchangeably with “amplification product,” refers to the nucleic acid molecule generated during an amplification procedure that is complementary or homologous to a sequence contained within the target sequence. These terms can be used to refer to a single strand amplification product, a double strand amplification product or one of the strands of a double strand amplification product.

The interchangeable terms “single nucleotide polymorphism”, “SNP” and “point mutation” refer to a nucleotide substitution in a single position in a nucleic acid sequence. As used herein, it may refer to one or more single nucleotide polymorphisms, and/or double point polymorphisms or triple point polymorphisms.

The terms “wild type” or “WT” as used herein refer to a starting nucleic acid sequence. It is not limited to naturally occurring sequences, and can refer include any starting nucleic acid sequence originating from any source including animals, plants, microorganisms, fungi, viruses and/or artificial starting strains.

The term “probing” as used herein refers to detection of nucleic acid sequence with a probe.

Furthermore, it should be understood that in the attached FIGS, 1 cycle corresponds to one 1 minute. The term “Rn” corresponds to the normalised reporter value. The term “ΔRn” corresponds to the Rn signal value minus the baseline Rn signal generated by the instrument.

### Fluorescent Probes

The probes used in the methods and kits of the present invention comprise an oligonucleotide probe sequence complementary to a region of a target nucleic acid sequence, wherein the oligonucleotide probe sequence has only one fluorophore ligand and which ligand is bound to an internal cytosine base and wherein said oligonucleotide probe sequence does not have a 3′ end terminator.

In some embodiments, the probe sequence is a DNA sequence. In some embodiments the target nucleic acid sequence is a DNA sequence. The fluorescence of the probe increases to indicate the presence of the target nucleic acid in a sample.

The cytosine base is preferably substantially centrally disposed along the oligonucleotide's length. Centrally disposed means between the third nucleotide from the 3′ end and the 5′ end. There are particular benefits associated with labeling the probe internally at a cytosine base. The probes are elongated and become incorporated into a nucleic acid, for example DNA, product during isothermal amplification, such as LAMP, which allows for performing a melt curve analysis on the generated product. In the probe, the fluorophore is conjugated to an internal cytosine complementary to guanine in the antisense strand. Guanine affects the excitation state of many fluorophores resulting in a formation of unique melt curve signatures and allows distinguishing between specific and unspecific products generated under isothermal conditions.

The use of a fluorophore ligand disposed on an internal cytosine base on probe sequences that become exited when base paired with guanine on their target sequence is particularly advantageous because complex two-part reaction setups comprising fluorophore-bound sequences and complementary quencher-bound sequences are not required. Such setups require laborious design protocols and optimisation such as designing alternative complementary regions for the fluorophore tagged sequences and quencher tagged sequences. The fluorescence will increase along with the growing amplicons in the present disclosure because the fluorophores are disposed on probes that become incorporated into the exponentially growing amplicons.

The oligonucleotide probe does not contain a ddNTP at its 3′ end. This enables incorporation of the labelled oligonucleotide into the amplicon. Thus, the 3′ end of the probe is not “blocked”.

The fluorophore may comprise any one or more selected from the following: FAM, JOE, TET, HEX, TAMRA, ROX, ALEXA and ATTO, or any other suitable fluorophore.

The probe may comprise the following sequence:

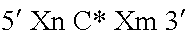

Where n is >1, m is >3, X is nucleotide base; and * is a fluorophore. Preferably, the nucleotide base may be selected from A, T, C and G. Preferably, n is more than 1 to 20 or less, more preferably more than 1 to 10 or less. Preferably, m is more than 3 to 20 or less, more preferably more than 3 to 10 or less. It is contemplated that all combinations of lengths of probe covered by the possible number of nucleotides that n or m make take by the preceding ranges are disclosed. For example, n may be more than 1 to 20 or less, more than 2 to 19 or less, more than 3 to 18 or less, more than 4 to 17 or less, more than 5 to 16 or less, more than 6 to 15 or less, more than 7 to 14 or less, more than 8 to 13 or less, more than 9 to 12 or less, more than 10 to 11 or less, or any combination thereof, and may be in conjunction with m being more than 3 to 20 or less, more than 4 to 19 or less, more than 5 to 18 or less, more than 6 to 17 or less, more than 7 to 16 or less, more than 8 to 15 or less, more than 9 to 14 or less, more than 10 to 13 or less, more than 11 to 12 or less, or any combination thereof.

The fluorescence is increased when the oligonucleotide probe is incorporated into the target nucleic acid sequence which results in a change in the configuration of the amplicon-probe complex leading to an alteration of the fluorophore excitation state.

The cytosine bound to the fluorophore ligand is not disposed at or next to the 5′ or 3′ end. More preferably it is not disposed in the first 3 bases from either the 5′ or 3′ end. Preferably, the cytosine bound to the fluorophore is positioned at least 3 bases from the 3′ end. Particularly preferably the cytosine bound to the fluorophore is disposed at the middle base of the probe.

### Target Nucleic Acid

The target nucleic acid may be a sequence obtained from a micro-organism, fungi, yeast, virus, human, animal, or plant, or any portion or variation thereof. The target nucleic acid for LAMP is known to enable LAMP primers and appropriately specific probes to be synthesized. Thus, the presence or absence of said micro-organism, fungi, yeast, virus, human, animal or plant sequence in a sample can be determined.

The fluorescence increases to indicate the presence of the target nucleic acid in a sample.

### Buffers

The reactions can be performed using buffering systems such as 0.1M Tris pH 7-9 (e.g., an RB or RB Tris buffer); 0.25 M CHES/CAPSO (95/5) pH 7-9 and 0.01M Tris pH 7-9 with 0.1% Tween 20 (MELT). Other similar buffers, or TE buffers at approximately pH 8 may also be used.

### Example 1

### Method

Detection of wild-type (WT) and mutant (SNP) sequences in a single reaction tube using 2 fluorescent probes was carried out as follows. The first probe is complementary to a WT sequence and the second probe is for a mutant sequence containing an SNP (SNP end sequence). The probes are labelled with different fluorophores: the WT probe is labelled with TAMRA (WT-TAMRA) and the second probe is labelled with Cy5 (SNPend-Cy5). The probes are designed based on one of the LAMP loop primer sequences (LF or LB) and the point mutation is located at the 3′ end of one of the probes. The reaction is performed using a standard RB (0.1 M Tris buffer, pH 8) buffer.

### Assay Set Up

Experiments were conducted using an artificial DNA sequence (gBlock™; WT) and a previously validated set of IC (internal control) LAMP primers (DNA sequences listed below). An artificial DNA sequence (gBlock™) with a single point mutation located at the 3′ end of LB primer (A to C) was ordered from Integrated DNA Technologies™ (IDT). The IC primer mix was prepared without loop primers (Table 1). The assay was set up using standard LAMP duplex pellets containing 16 units BST DNA polymerase per reaction, dNTPs and buffering agents.

### Assay Procedure

A reaction master mix was prepared by reconstituting 10×LAMP duplex pellet in 20 μl of 5× buffer (0.5 M Tris, pH 8); 38 μl of primer probe mix and 32 μl of H20.

9 μl of master mix and 1 μl of WT or SNP end DNA (at 1 pg/μl) was used per reaction. For NTC (no template control) reactions, the reaction mixture was topped up with 1 μl of molecular grade water. WT and SNP end DNA samples were tested in separate reactions or in the same tube in duplicates for 60 minutes at a constant temperature of 63° C. using the ABI7500 instrument.

TABLE 1
Preparation of primer mix for SNP studies

| IC Primer | Volume [μl] |
|---|---|
| 250 μM Fip primer | 6.4 |
| 250 μM Bip primer | 6.4 |
| 250 μM F3 primer | 1.6 |
| 250 μM B3 primer | 1.6 |
| Loop F primer | — |
| Loop B primer | — |

TABLE 2
Preparation of primer probe mix duplex pellet for LAMP
SNP studies using competitive WT and SNP end fluorescent
probes labelled with different fluorophores.

| Component (10 reactions) | Volume [μl] for 10rxn (1 pellet) |
|---|---|
| IC primer mix | 0.356 |
| IC-TAMRA probe (1 uM) | 12.6 |
| (WT-TAMRA probe) | |
| IC SNP2-CY5 probe (1 uM) | 25.2 |

The LAMP master mixed used for the assay was:
1 duplex pellet (sufficient for 10 reactions)
38 μl primer+probe
20 μl 5×RB buffer (0.5 M Tris, pH 8)
32 μl H₂O

### Results

Using WT-TAMRA and SNP-Cy5 probes in the same reaction enabled distinction of WT and SNP sequences which differed by a single nucleotide. WT and SNP end sequences were distinguishable using their corresponding WT or SNP end probes when the experiment was carried out in the same vessel (multiplex).

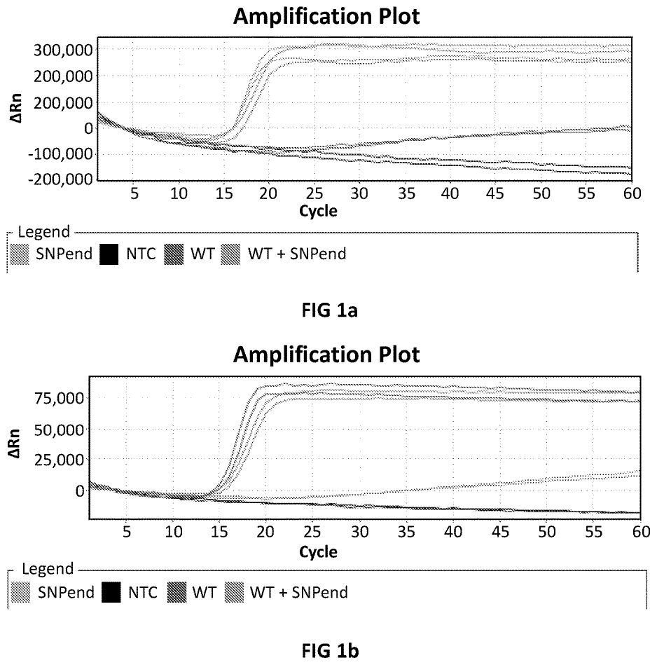

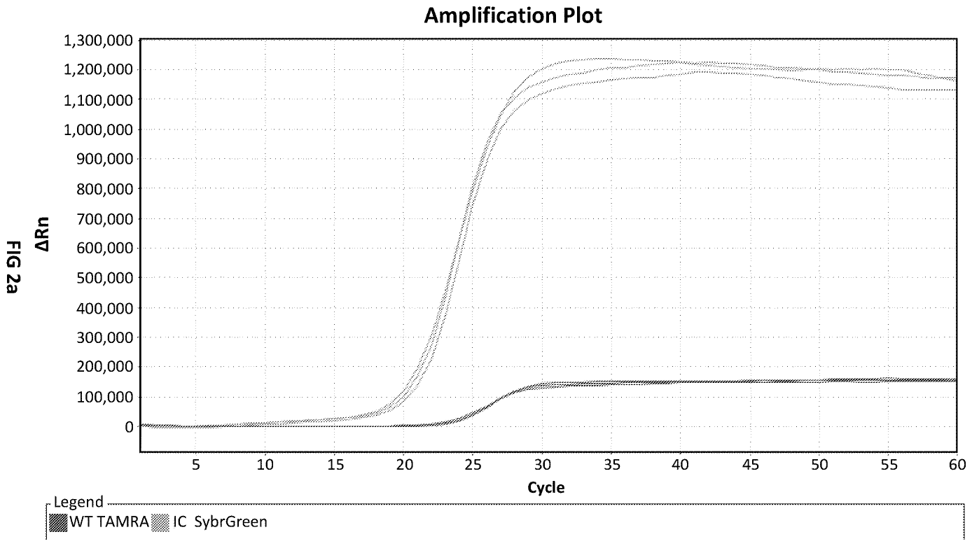

FIG. 1a is an amplification plot showing the changes in fluorescence in the Cy5 channel in LAMP reactions each comprising a DNA sample which is either an SNP end sequence complementary to the SNP end probe (SNP end), a WT sample (differing from the SNP end sample by a single nucleotide) and complementary to WT probe, a sample comprising both WT and SNP end sequences, or an NTC reaction.

As shown in FIG. 1a, significant rises in fluorescence were detected during the LAMP reaction when DNA comprising an SNP end sequence was present in the sample. In contrast, when only a WT sequence was present, no significant increase in fluorescence was observed. Thus, a DNA sequence comprising an SNP could be differentiated from a WT DNA sequence.

FIG. 1b shows the changes in fluorescence in the TAMRA channel in LAMP reactions each comprising a DNA sample which was either an WT sequence complementary to the WT probe (WT), a SNP end sequence (differing from the WT sequence by a single nucleotide), complementary to SNP probe, a sample comprising both WT and SNP end sequences, or an NTC reaction.

As shown in FIG. 1b, significant rises in fluorescence during the LAMP reaction occurred when a DNA sample comprising a WT sequence was present in the sample. In contrast, when only an SNP end sequence was present, no significant increase in fluorescence was observed. Thus, a DNA sequence comprising an SNP could be differentiated from a WT DNA sequence. Table 3 shows the amplification times or CT values (cycle threshold) defined as the number of cycles required for the fluorescent signal to cross the threshold (i.e., exceeds background level), for each of the experiments performed.

TABLE 3
Amplification times (CT values) generated in the presence
of WT and SNP end DNA with WT-TAMRA and SNP end-Cy5 probes.

| | Sample/ | WT-TAMRA | SNP end-CY5 |
|---|---|---|---|
| Tube | gBlock DNA | probe CT value | probe CT values |
| 1 | WT | 15.98 | — |
| 2 | WT | 16.58 | — |
| 3 | SNPend | — | 16.61 |
| 4 | SNPend | — | 16.73 |
| 5 | WT + SNPend | 17 | 17.13 |
| 6 | WT + SNPend | 17.54 | 17.92 |
| 7 | NTC | — | — |
| 8 | NTC | — | — |

### Sequences:

C-position of the fluorophore
A-3′ end
SEQ ID NO. 4: WT sequence:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

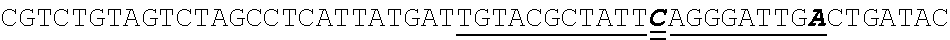

CGGAAGACATCTCAAATGAAGTGGTCTATGCGACAGAGAC
SEQ ID NO. 5: WT probe label:

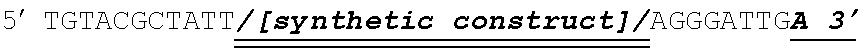

SEQ ID NO. 44: WT probe labelled with TAMRA:

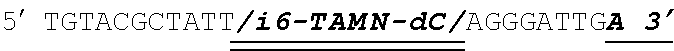

SEQ ID NO. 6: SNP end (end A to C) sequence:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

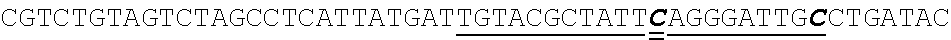

CGGAAGACATCTCAAATGAAGTGGTCTATGCGACAGAGAC
SEQ ID NO 7: SNP end (A to C) probe label:

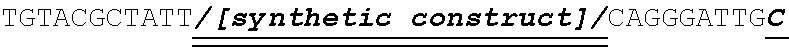

SEQ ID NO 45: SNP end (A to C) probe labelled with Cy5:

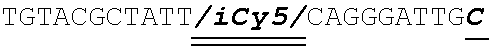

Wild type IC LAMP primers:
SEQ ID NO. 8: F3
GAGACCACTGACCGATCTA
SEQ ID NO. 9: B3
GAAGTGGTCTATGCGACAG
SEQ ID NO. 10: LoopF
CGGAGAGATAACTACGGTGC
SEQ ID NO. 11: LoopB
TGTACGCTATTCAGGGATTGAC
SEQ ID NO. 12: FIP
GACAGAGGGGCTGTAAGCGGACCATCTGTGTGGTACTG
SEQ ID NO. 13: BIP
CGCTGACGTCTGTAGTCTAGCTGAGATGTCTTCCGGTATCA

### Example 2

### Method

The method for distinguishing between WT and SNP sequences in a single reaction tube using a first fluorescent probe and a blank oligo was conducted as follows:

The probes used were as described in Example 1. Three different types of experiment (Experiments 2a, 2b and 2c) were performed. Each experiment utilised a probe complementary to a WT DNA sequence which was labelled with a fluorophore (WT-TAMRA), and an intercalating dye (SybrGreen) for detecting both WT and SNP sequences. The WT-TAMRA probe only generated a fluorescence signal when a WT sequence was present in the sample, whereas the intercalating dye (SybrGreen) served as a control which detected both the WT and SNP sequences. The WT probe was designed based on the sequence of one of the loops (LF or LB) and the point mutation(s) were located to be positioned opposite to:
a) the 3′ end of the probe (Experiment 2a)—SNP end (A to C) probe
b) second base from the 3′ end of the probe (Experiment 2b)—BO S1 T probe (G to T)
c) for a double mutation: one at 3′ end of the probe and another one on second base from 3′ end of the probe (Experiment 2c)—BO S1 SNP2 C Blank Oligo

A DNA blank oligo was also present in the reaction. The blank oligo had the same sequence as the fluorescent probe, but it was not labelled with a fluorophore, and comprised one or more SNP mutation(s). During the LAMP reaction a blank oligo competes with the fluorescent probe for the binding site on the amplified nucleic acid, which increases the specificity of detection.

### Assay Set Up:

The LAMP primer mix was as described in Example 1, and prepared as described in Tables 1 and 2, and was diluted 1:10. The composition of the LAMP master mix was as follows:
1 duplex pellet (sufficient for 10 reactions)
20 μl of 5×RB tris buffer
12.6 μl of WT-TAMRA probe (1 μM)
12.6 μl of a blank oligo (1 μM)—
   SNP end (A to C) blank oligo;
   BO S1 T (G to T) blank oligo; or
   BO S1 SNP2 C Blank Oligo;
3.56 μl IC primer mix (1:10);
1 μl SybrGreen (1:200);
40.24 μl H₂O.

### Assay Procedure:

Test samples for each experiment were prepared by adding 1 μl of the template DNA (WT, SNP end, S1 T or SNP2 C) at 1 pg/μl to 9 μl of the LAMP master mix. Each reaction was run in triplicates at 63° C. for 60 minutes using ABI7500 instrument. TAMRA and SybrGreen signals were detected in a single tube reaction.

### Results

### Control—No Blank Oligo

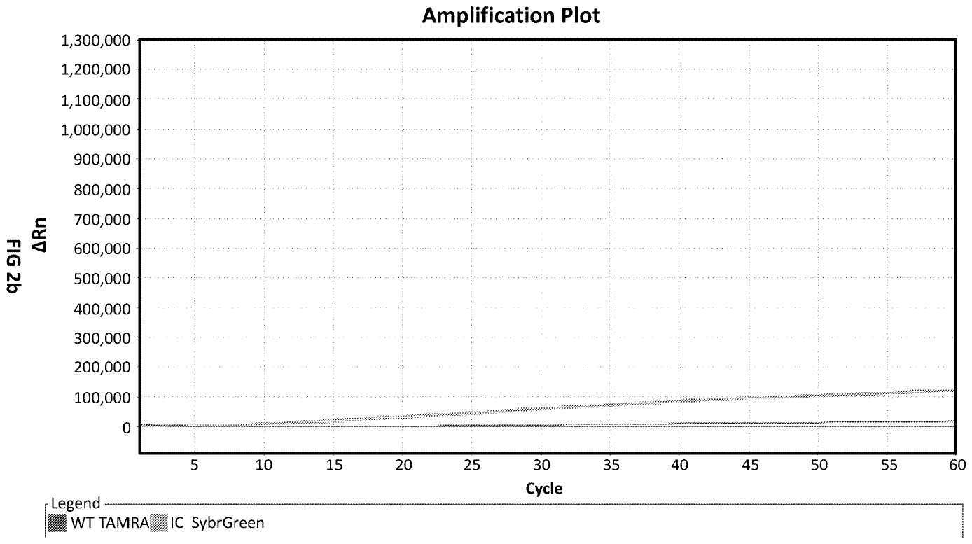

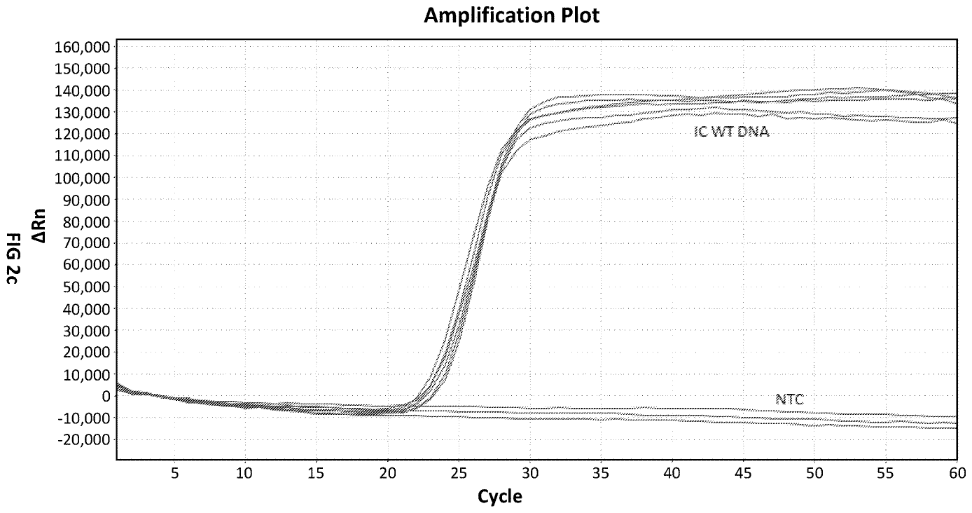

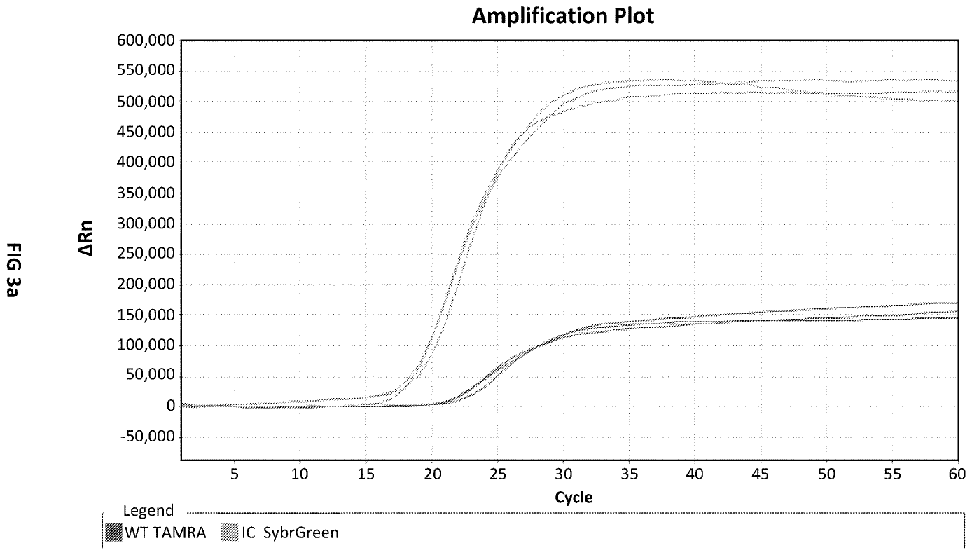

FIGS. 2a to 2c show amplification plots of a LAMP assay which comprised a WT fluorescent probe (WT-TAMRA), SybrGreen intercalating dye and WT sample (FIG. 2a), or that were NTC (no template control) reactions (FIG. 2b). The reactions did not comprise blank oligos or mutant DNA. FIGS. 2a to 2c show that both the WT probe and SybrGreen showed an increase in fluorescent signal in the presence of a WT DNA sample as the amplification reaction progressed, but did not show any increase in signal when a sample was not present. FIG. 2a shows outputs from SybrGreen and TAMRA channels for positive control samples (WT DNA) in the same graph, whereas FIG. 2c shows outputs from TAMRA channel only for positive controls and NTCs.

TABLE 4
Results of control assays using WT-TAMRA; no Blank Oligo.

| | WT DNA | No template control (H2O) |
|---|---|---|
| WT - TAMRA | Detected | Not detected |
| SybrGreen control | Detected | Not detected |

Experiment 2a—SNP End (A to C) Blank Oligo

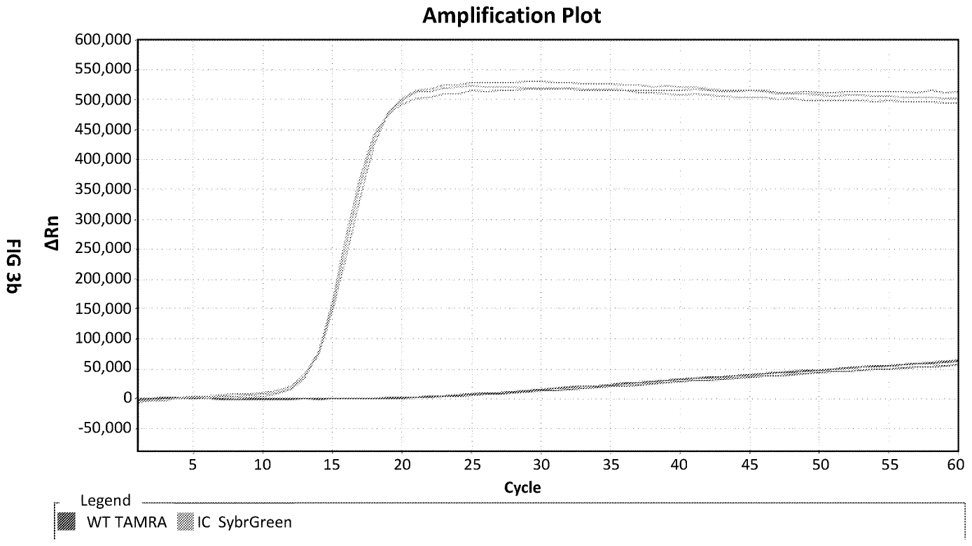

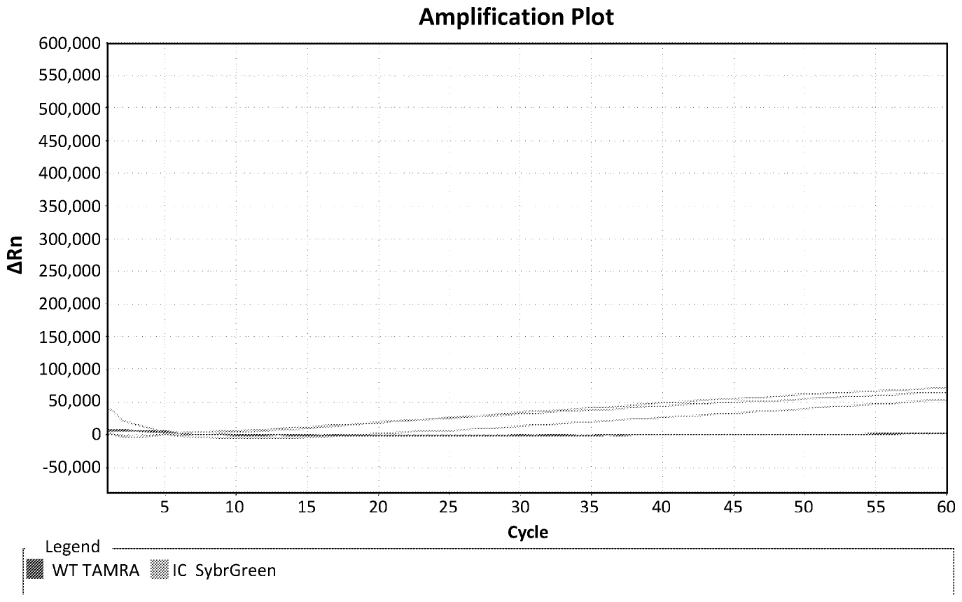

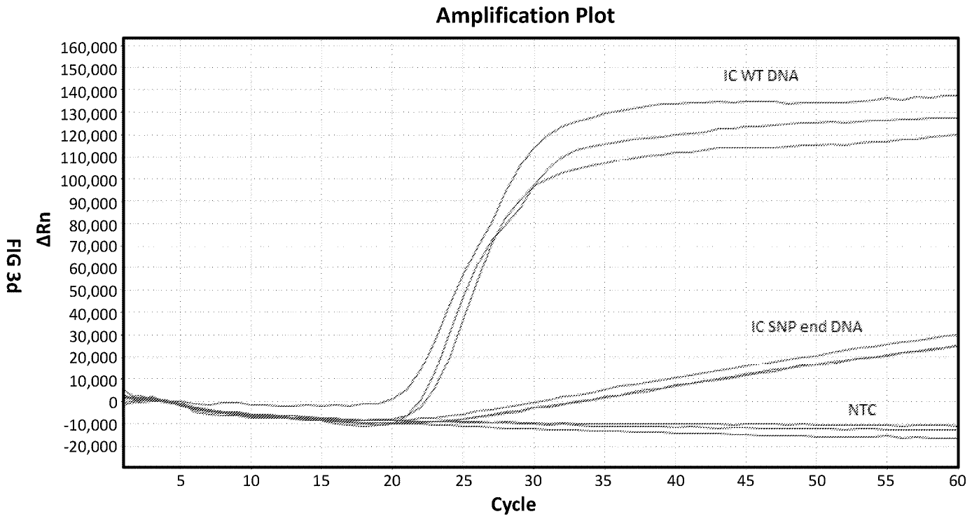

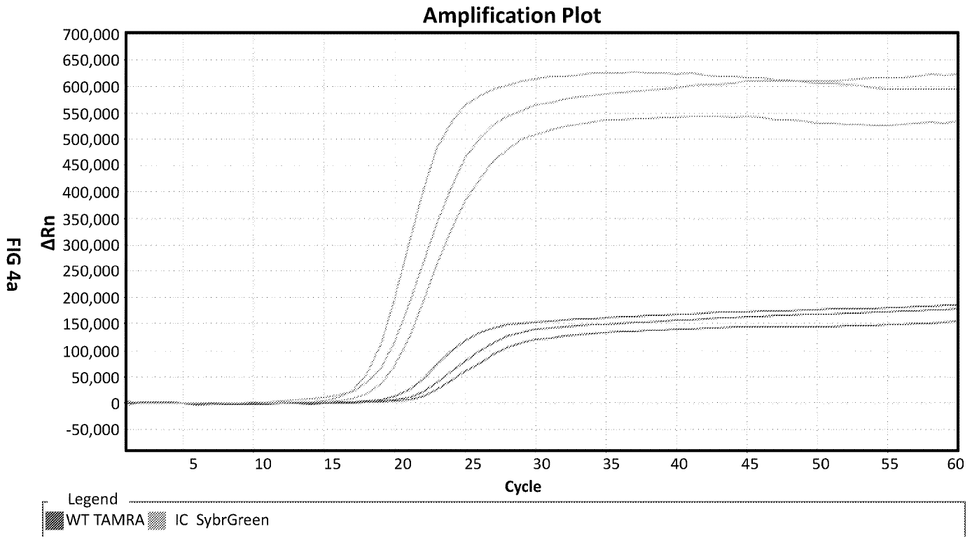

FIGS. 3a to 3d show LAMP experiments conducted according to a method of the invention in the presence of a fluorescent WT probe, an intercalating dye and a SNP end blank oligo (having a SNP at the 3′ end) as described above. As shown in FIG. 3a, in the presence of a sample comprising a WT sequence, both the probe and the intercalating dye displayed an increase in signal as the LAMP reaction progressed, because both fluorescent components were able to bind to the amplified DNA.

However, as shown in FIG. 3b, in the presence of a sample which only comprises an SNP end sequence (which differs from the WT sequence by a single nucleotide), only the intercalating dye showed any significant increase in signal. Thus, the combination of a probe and blank oligo was able to distinguish and detect WT and SNP-containing sequences. FIG. 3c is a no template control (NTC) experiment, which showed no significant increase in fluorescence when a sample was not present. FIG. 3d shows a comparison of output signals in the TAMRA channel in the presence of WT and SNP-containing sequences.

TABLE 5
showing the results of Experiment 2a, using a WT -
TAMRA probe and a SNP end (A to C) blank oligo.

| | | IC SNP end | No template |
|---|---|---|---|
| | IC WT DNA | (A to C) DNA | control (H₂O) |
| WT - TAMRA | Detected | Not detected | Not detected |
| SybrGreen control | Detected | Detected | Not detected |

Experiment 2b—S1 T (G to T) Blank Oligo

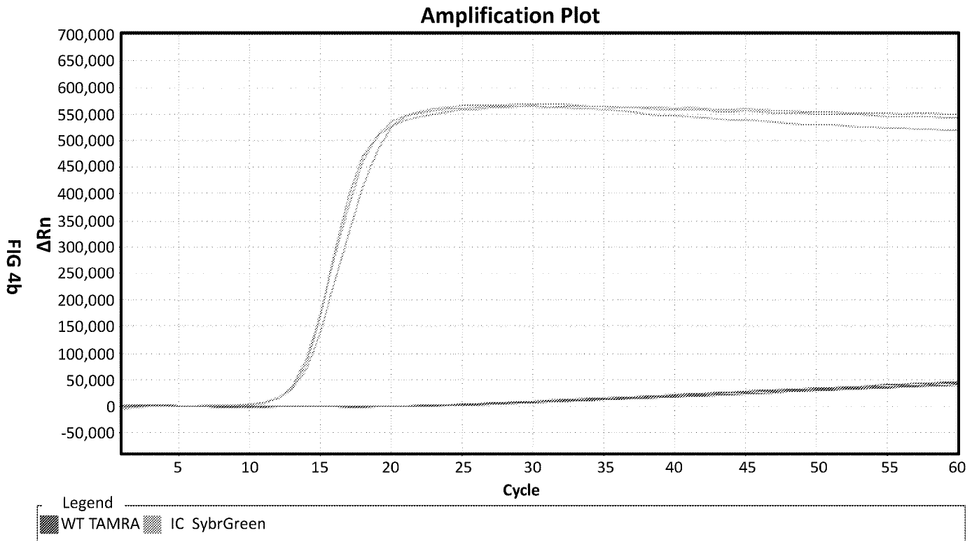

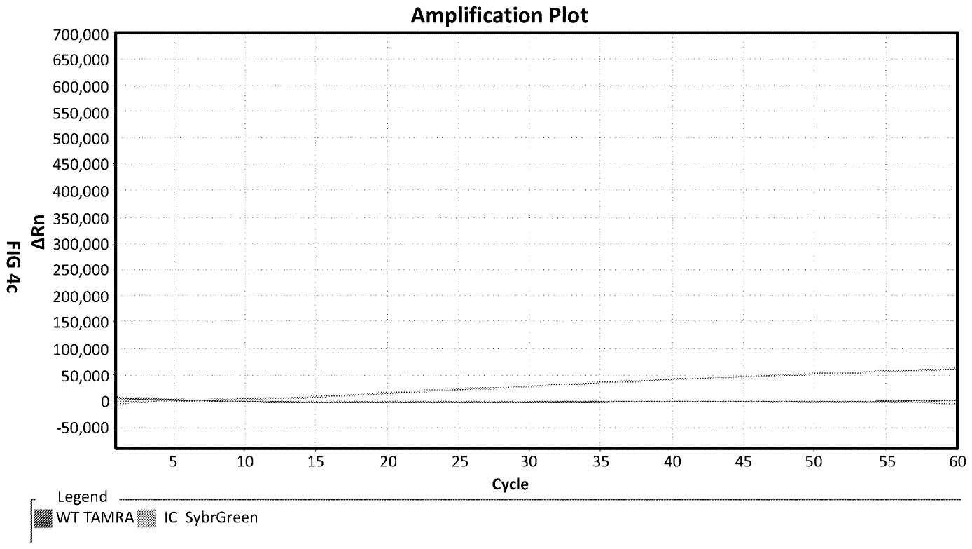

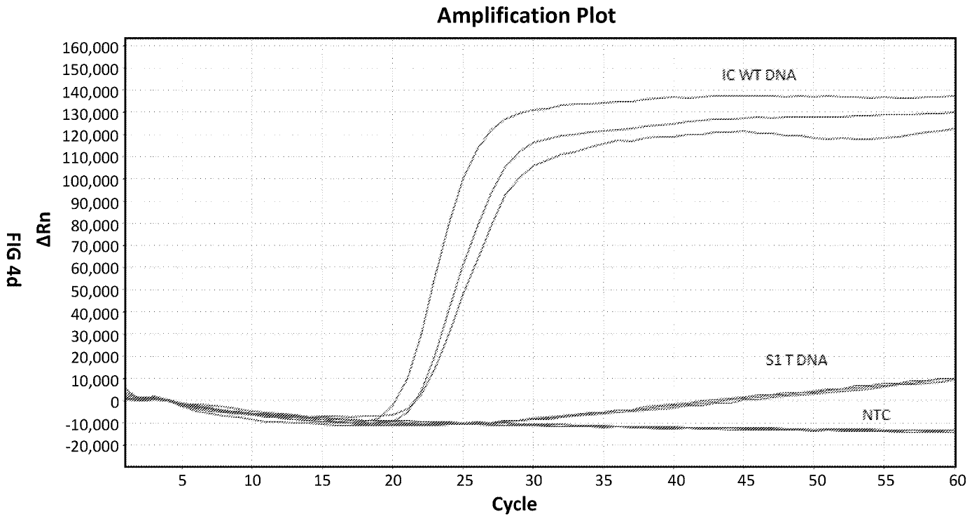

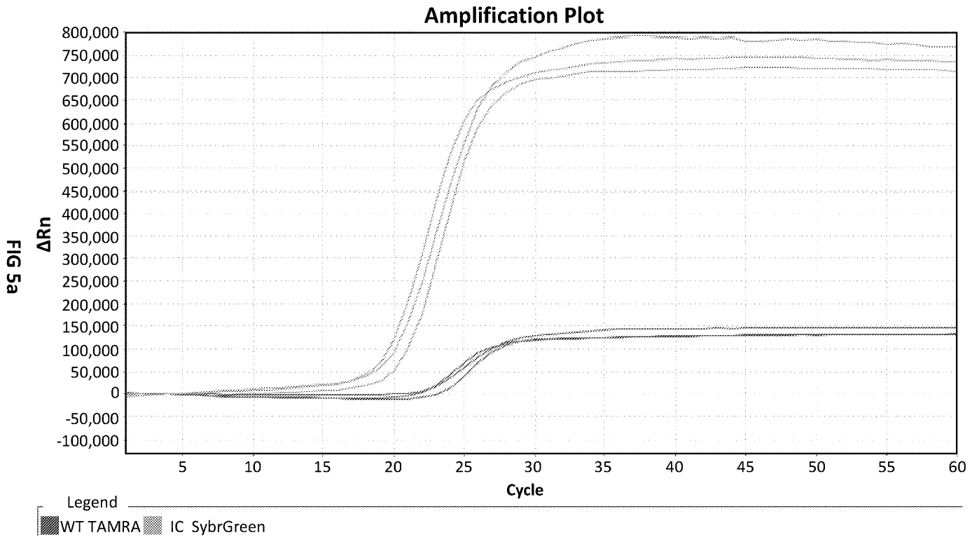

FIGS. 4a to 4d show LAMP experiments conducted according to a method of the present invention in the presence of a fluorescent WT probe, an intercalating dye and a S1 T (G to T) blank oligo (which has an SNP on the second nucleotide from the 3′ end) as described above. As shown in FIG. 4a, in the presence of a WT sequence in a sample, both the probe and the intercalating dye displayed an increase in signal as the LAMP reaction progressed, because both fluorescent components were able to bind to the amplified DNA.

However, as shown in FIG. 4b, in the presence of a sample which only comprises an S1 T (G to T) sequence (which differs from the WT sequence by a single nucleotide), only the intercalating dye showed any significant increase in signal. Thus, the combination of a probe and blank oligo was able to distinguish and detect WT and SNP-containing sequences, independently of whether the SNP was at the nucleotide at the 3′ end, or the second nucleotide from the 3′ end. FIG. 4c is a no template control (NTC) experiment, which showed no significant increase in fluorescence when a sample was not present. FIG. 4d shows a comparison of output signals in the TAMRA channel in the presence of WT and SNP-containing sequences.

TABLE 6
showing the results of Experiment 2b, using a
WT - TAMRA probe and S1 T (G to T) blank oligo

| | | S1 T | No template |
|---|---|---|---|
| | IC WT DNA | (G to T) DNA | control (H₂O) |
| WT - TAMRA | Detected | Not detected | Not detected |
| SybrGreen control | Detected | Detected | Not detected |

Experiment 2c—S1 SNP2 C Blank Oligo

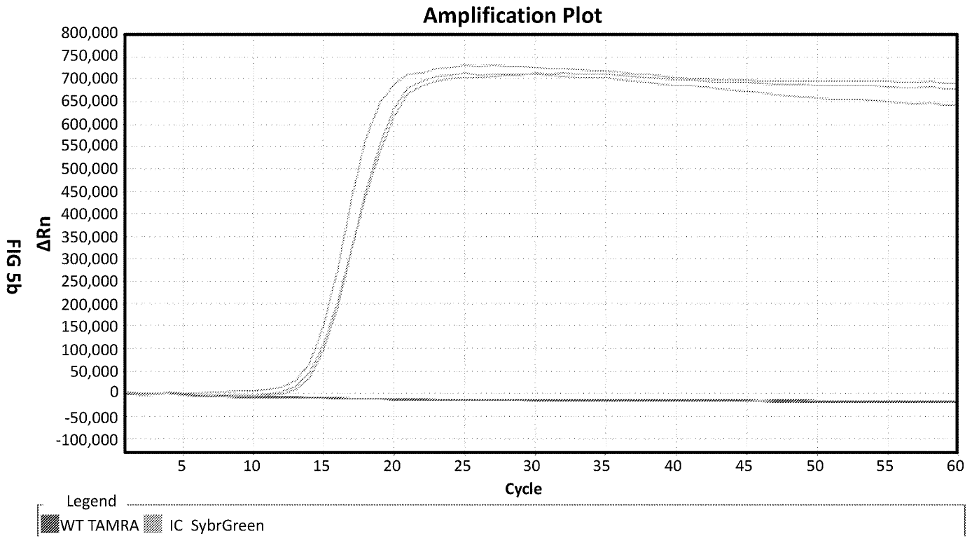

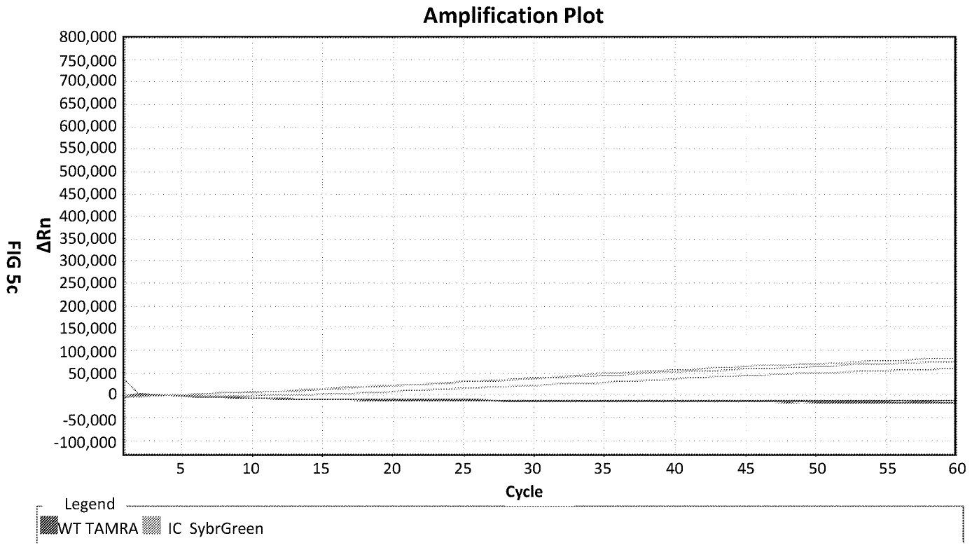

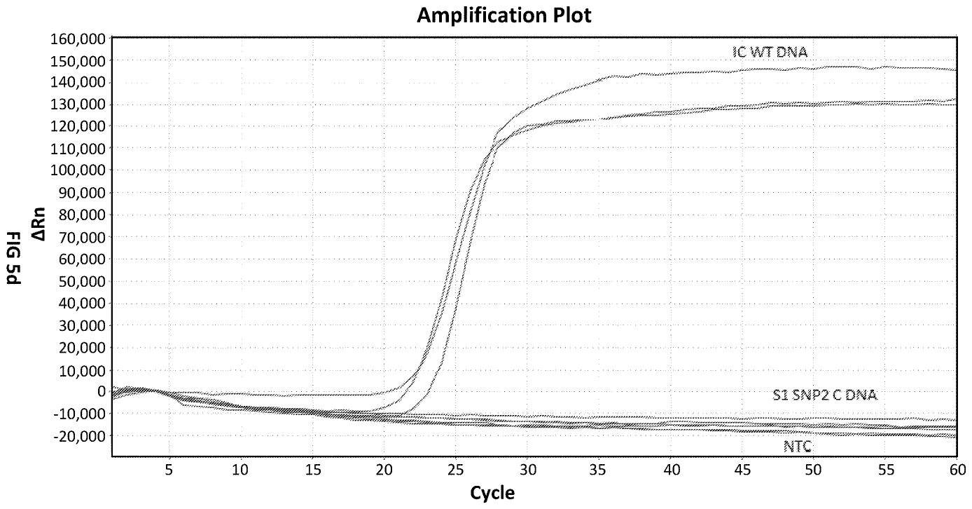

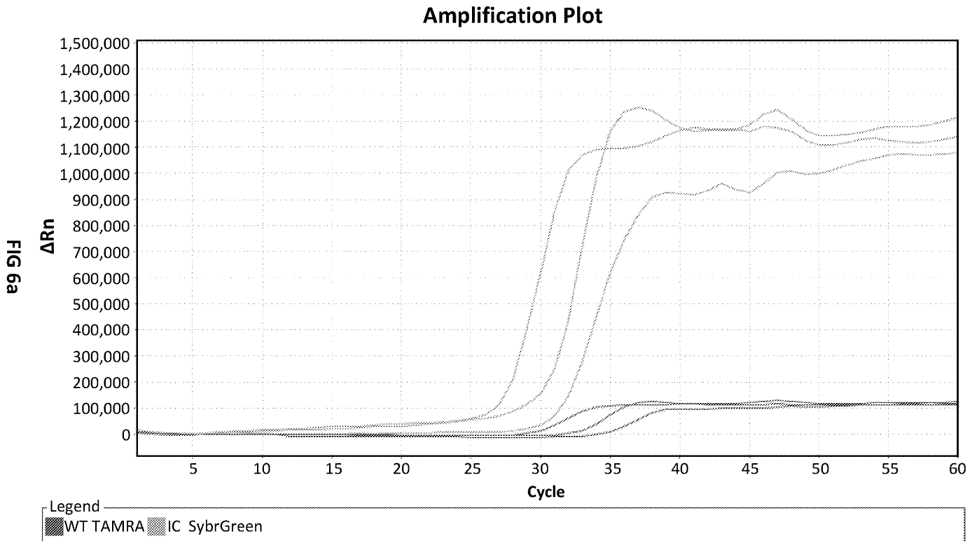

FIGS. 5a to 5d show LAMP experiments conducted according to a method of the invention in the presence of a fluorescent WT probe, an intercalating dye and a S1 SNP2 blank oligo (which comprises a double-point mutation on the first and second nucleotides from the 3′ end) as described above. As shown in FIG. 5a, in the presence of a WT sequence in a sample, both the probe and the intercalating dye displayed an increase in signal as the LAMP reaction progressed, because both fluorescent components were able to bind to the amplified DNA.

However, as shown in FIG. 5b, in the presence of a sample which only comprises an S1 SNP2 C sequence (which differed from the WT sequence by two consecutive nucleotides), only the intercalating dye showed any significant increase in signal. Thus, the combination of a probe and blank oligo was able to distinguish and detect WT and SNP-containing sequences, independently of whether the mutation comprises one or two nucleotides in a sequence. FIG. 5c is a no template control (NTC) experiment, which showed no significant increase in fluorescence when a sample was not present. FIG. 5d shows a comparison of output signals in the TAMRA channel in the presence of WT and SNP-containing sequences.

TABLE 7
showing the results of Experiment 2, using a
WT - TAMRA probe and S1 SNP2 C blank oligo

| | | S1 SNP2 | No template |
|---|---|---|---|
| | IC WT DNA | C DNA | control (H₂O) |
| WT - TAMRA | Detected | Not detected | Not detected |
| SybrGreen control | Detected | Detected | Not detected |

Control—without Blank Oligos

### Example 2d

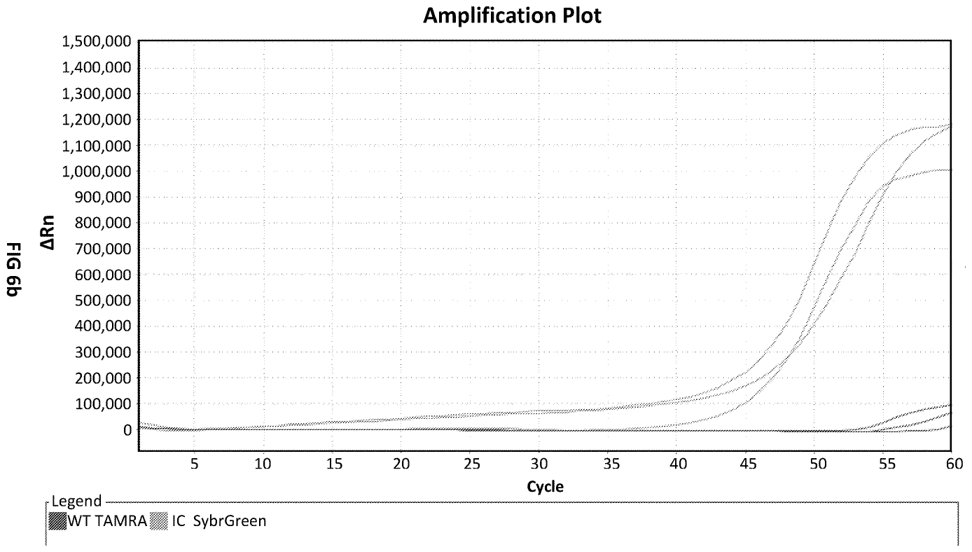

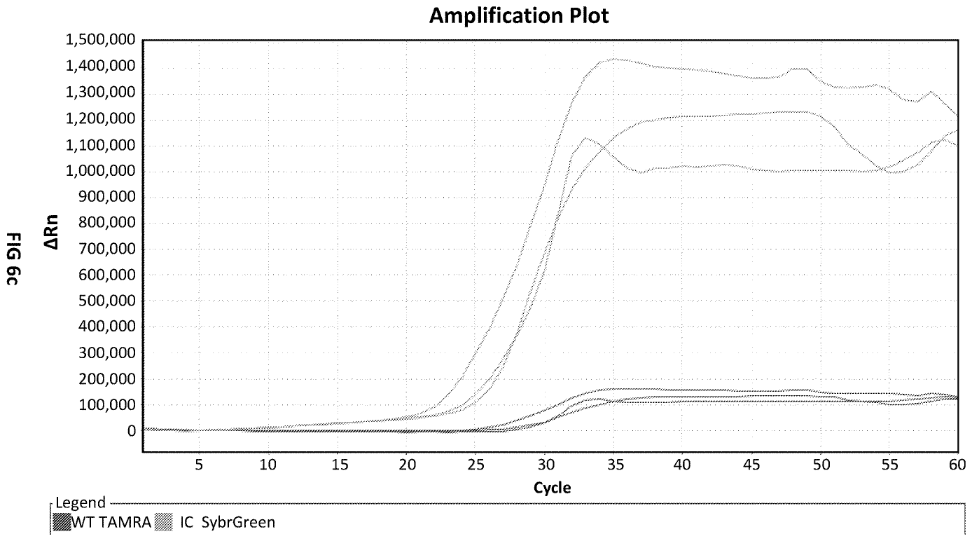

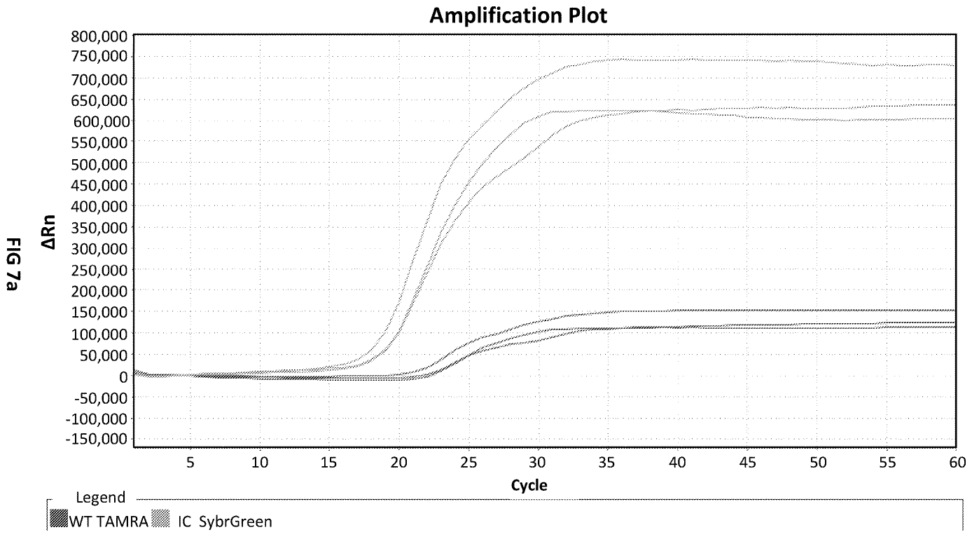

Experiments 2a-2c were repeated using artificial DNA sequences (SNP end DNA, S1 T DNA and S1 SNP2 C DNA) not in the presence of blank oligos (FIGS. 6a to 6c). These experiments revealed that not adding a blank oligo to the reaction has a negative impact on reaction specificity. For example, FIGS. 6a to 6c show that without blank oligos the fluorescent WT probe was able to detect SNP sequences. This was in contrast to the results obtained from Experiments 2a-2c. In Experiments 2a-2c, blank oligos were part of the assay and the fluorescent WT probe generated an increase in signal only in the presence of WT DNA but not SNP-containing variants. These results indicate that incorporation of one or more blank oligo into the reaction allowed for specific detection of point mutations using fluorescent probe technology.

### Sequences:

C-position of the fluorophore
A-SNP mutation at or near 3′ end
SEQ ID NO. 14: WT sequence:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

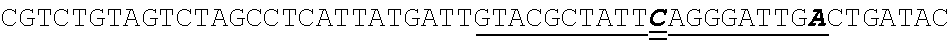

CGGAAGACATCTCAAATGAAGTGGTCTATGCGACAGAGAC
SEQ ID NO. 15: WT probe:

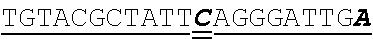

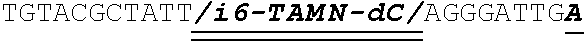

Experiment 1
SEQ ID NO. 16: SNP end (A to C) sequence:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

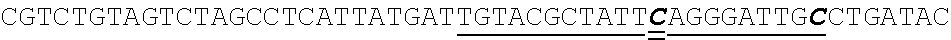

CGGAAGACATCTCAAATGAAGTGGTCTATGCGACAGAGAC
SEQ ID NO. 17: SNP end (A to C) Blank Oligo:

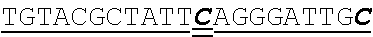

Experiment 2
SEQ ID NO. 18: S1 T (control)
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

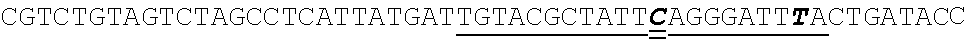

GGAAGACATCTCAAATGAAGTGGTCTATGCGACAGAGAC
SEQ ID NO. 19: BO S1 T (G to T) Blank Oligo:
TGTACGCTATTCAGGGATTTA
Experiment 3
SEQ ID NO. 20: S1 SNP2 C
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

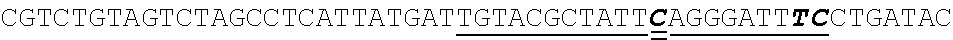

GGAAGACATCTCAAATGAAGTGGTCTATGCGACAGAGAC
SEQ ID NO. 21: BO S1 SNP2 C Blank Oligo
TGTACGCTATTCAGGGATTTC

### Example 3

### Method

A method for distinguishing between WT and SNP sequences in a single reaction tube using a fluorescent probe and a blank oligo, wherein the probe and blank oligo are designed such that the SNP is in the middle of the sequence was conducted as follows:

The LAMP master mix and reaction conditions were used as described in Example 2. Experiments were carried out using two sets of DNA, samples, probes and blank oligos comprising SNPs in the middle portion of the probe and blank oligo sequences. An SNP internal A to T set and SNP internal C to T set were used in experiments with an RB (Tris) buffer (Experiment 3a and 3b) and a CHES CAPSO buffer (Experiment 3c and 3d). The two sets differed in that the SNP mutation on the SNP internal C to T set was located at the nucleotide complementary to cytosine residue labelled with the TAMRA fluorophore on the fluorescent probe, while the SNP in the SNP internal A to T was located at a different position.

### Assay Set Up and Procedure

The LAMP primer mix and master mixes were prepared, and the assays carried out as described above in relation to Example 2.

### Results

### Experiment 3a

### RB (Tris) Buffer (SNP Internal C to T)

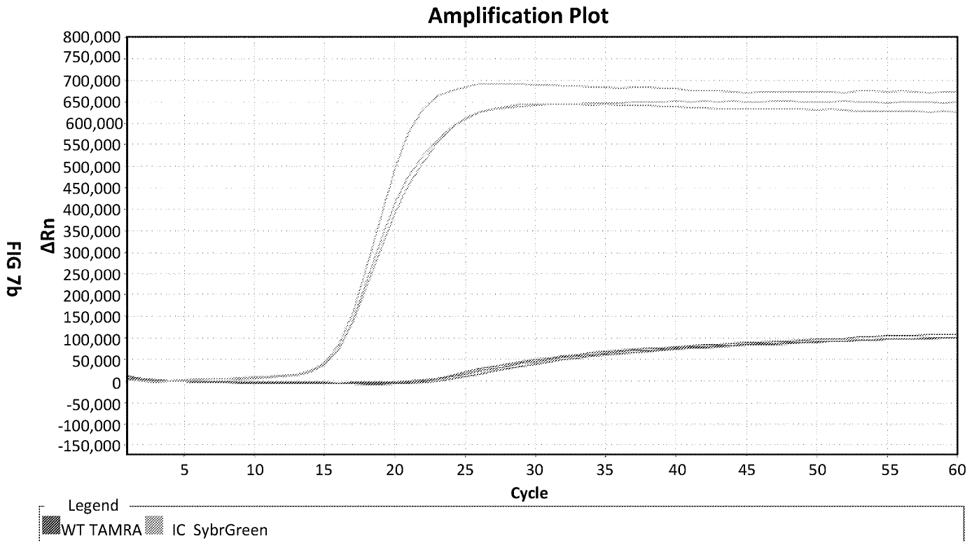

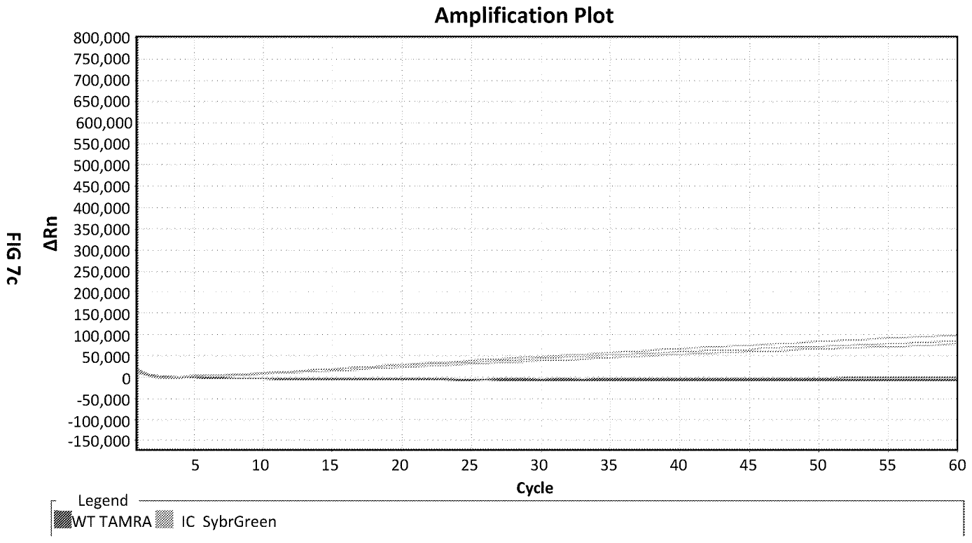

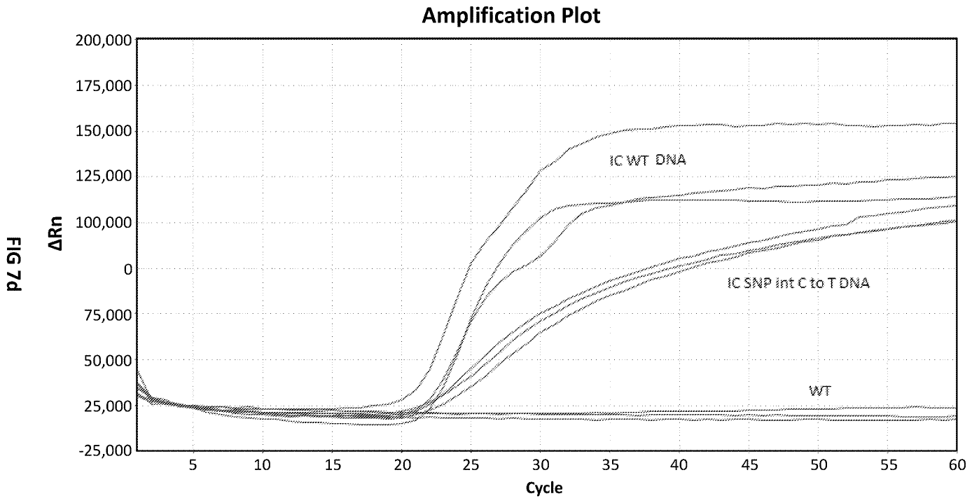

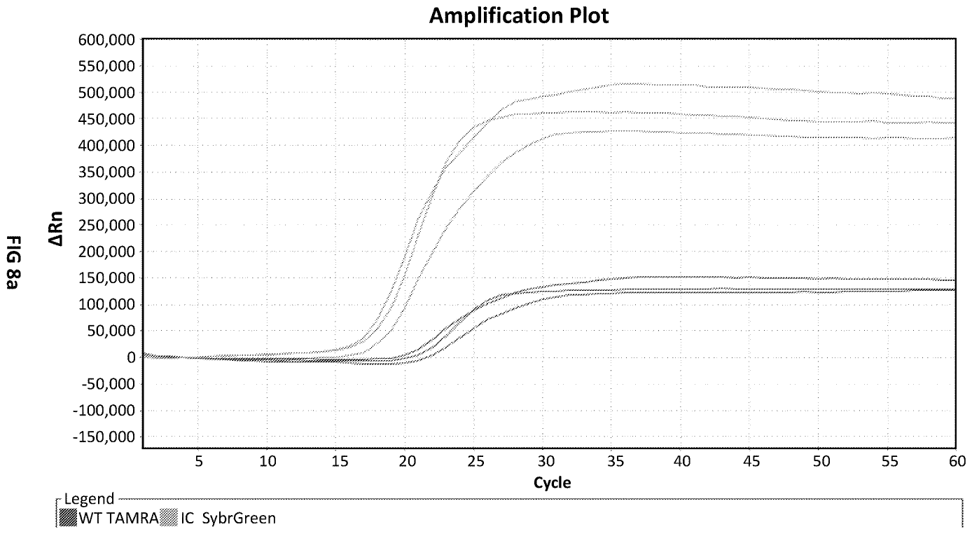

FIG. 7a shows an amplification plot of a LAMP reaction in a method which utilises a WT fluorescent probe, an SNP internal C to T blank oligo, SybrGreen intercalating dye, and a WT DNA sample. Both the probe and dye showed a significant increase in signal as the reaction progresses. FIG. 7b shows a corresponding plot for the SNP internal C to T DNA sample. While the WT probe signal is less strong than the signal for the WT sample, FIG. 7b showed a significant increase in signal for both the WT probe and the intercalating dye, indicating that there was a significant level of binding between the WT probe and the binding sites in the present in the SNP internal C to T DNA sample amplification. FIG. 7c shows an NTC experiment that showed no significant increase in signal for either fluorophore, in-line with previous experiments. FIG. 7d shows a comparison of output signals in the TAMRA channel in the presence of WT and SNP-containing sequences.

TABLE 8
results for SNP internal C to T under RB (tris) Buffer conditions

| | | IC SNP int | No template |
|---|---|---|---|
| | IC WT DNA | C to T DNA | control (H₂O) |
| WT - TAMRA | Detected | Detected | Not detected |
| SybrGreen control | Detected | Detected | Not detected |

### Experiment 3b

### RB (Tris) Buffer (SNP Internal a to T)

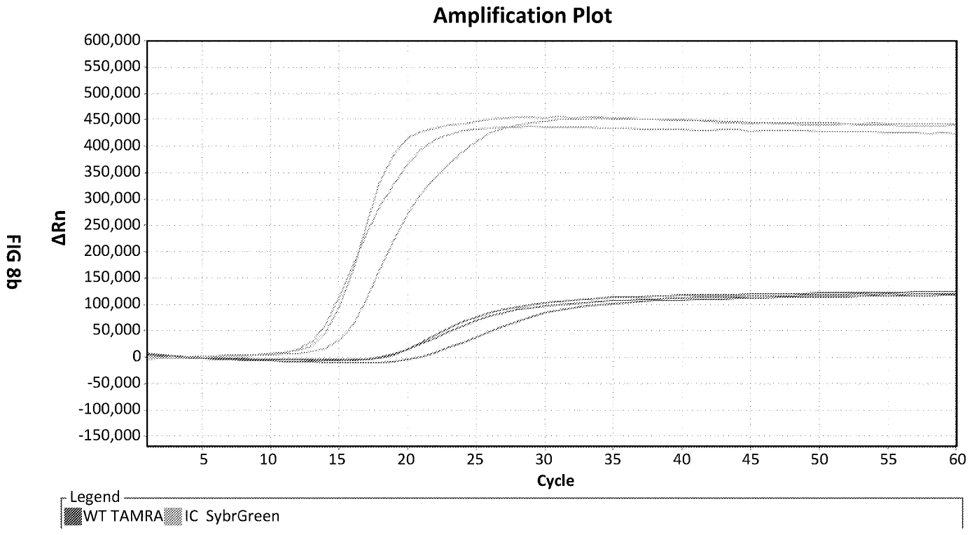

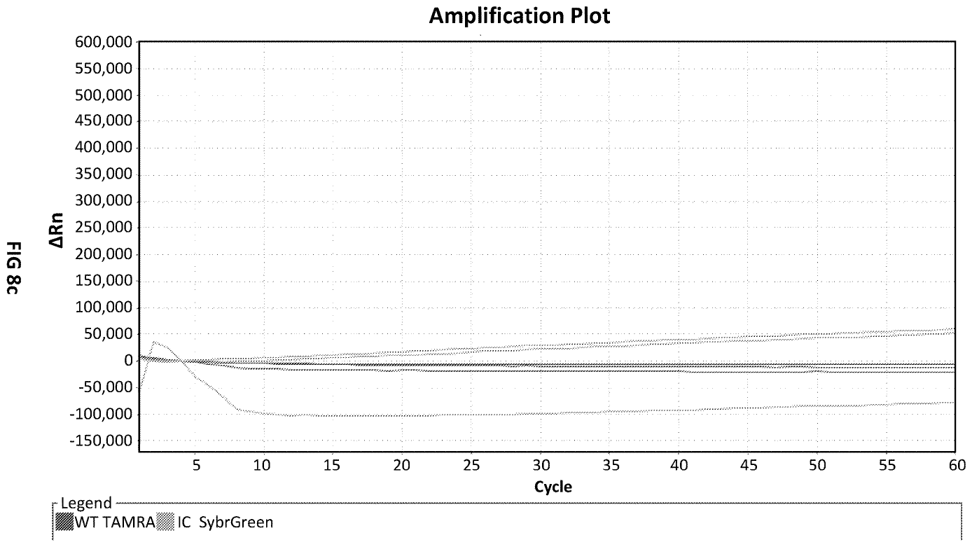

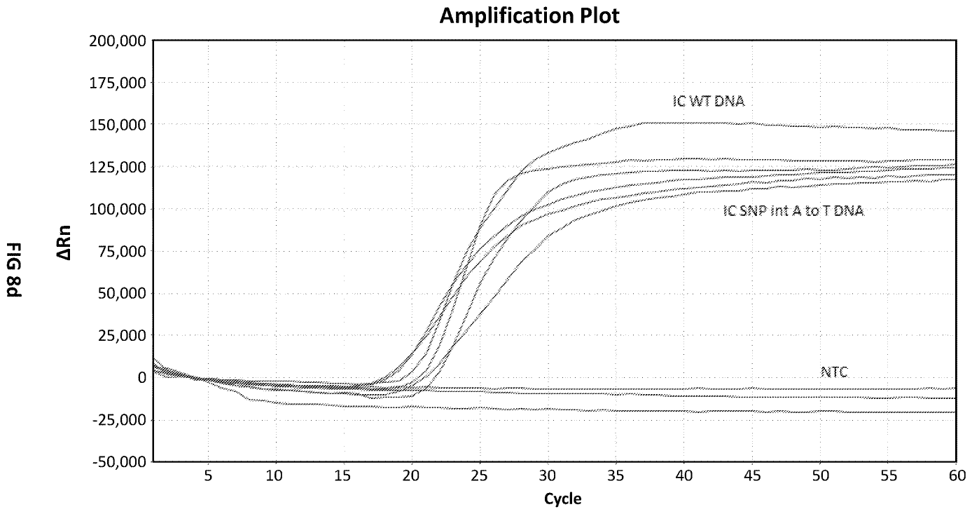

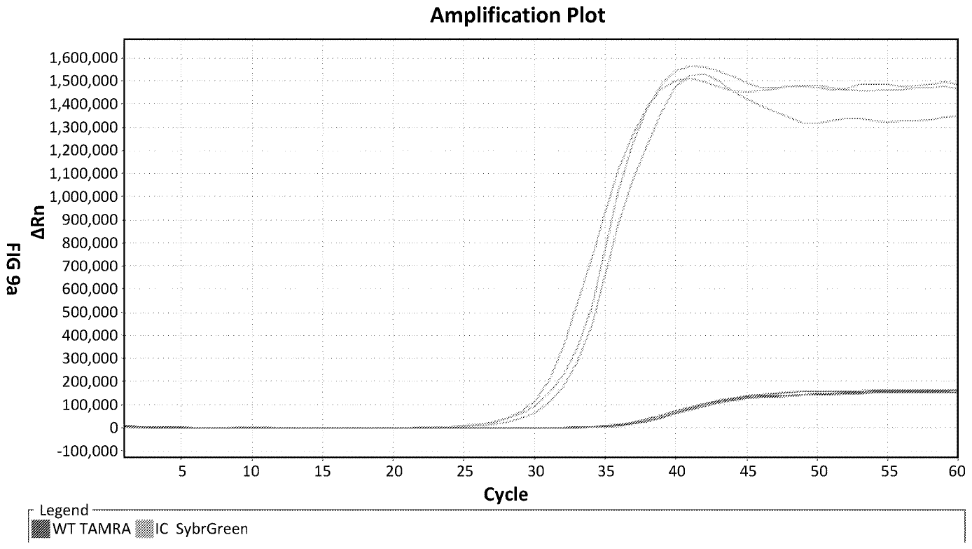

FIG. 8a shows an amplification plot of a LAMP reaction in a method which utilises a WT fluorescent probe, an SNP internal A to T blank oligo, SybrGreen intercalating dye, and a WT DNA sample. Both the probe and dye showed a significant increase in signal as the reaction progresses. FIG. 8b shows a corresponding plot for the SNP internal A to T DNA sample. While the WT probe signal is less strong than the signal for the WT sample, FIG. 8b showed a significant increase in signal for both the WT probe and the intercalating dye, indicating that there was a significant level of binding between the WT probe and the binding sites in the present in the SNP internal A to T DNA sample amplification. FIG. 8c shows an NTC experiment that showed no significant increase in signal for either fluorophore, in-line with previous experiments. FIG. 8d shows a comparison of output signals in the TAMRA channel in the presence of WT and SNP-containing sequences.

TABLE 9
results for SNP internal A to T under RB (tris) Buffer conditions

| | | IC SNP int | No template |
|---|---|---|---|
| | IC WT DNA | A to T DNA | control (H₂O) |
| WT - TAMRA | Detected | Detected | Not detected |
| SybrGreen control | Detected | Detected | Not detected |

Experiment 3c—CHES CAPSO Buffer (SNP Internal C to T)

While the specificity of SNP detection is reduced if the SNP is located in the middle portion of the blank oligo in an RB (tris) buffer environment (see Experiment 3a and 3b), the inventors have unexpectedly found that the specificity can be improved by altering the buffering conditions.

Specifically, experiments using the same conditions as Experiment 3a, and using SNP internal C to T and SNP internal A to T samples and blank oligo sets were performed in the presence of CHES CAPSO buffer, which was shown before to improve assay specificity.

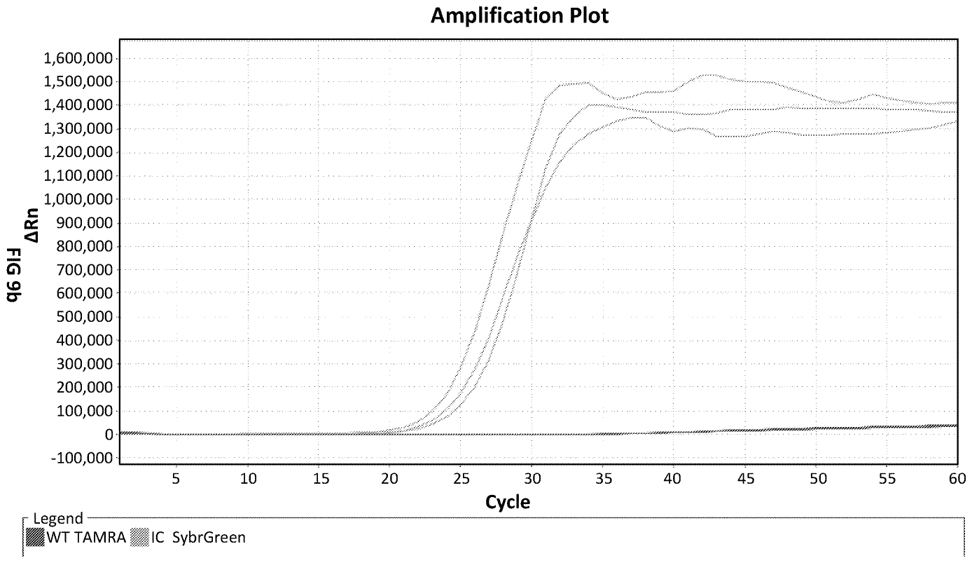

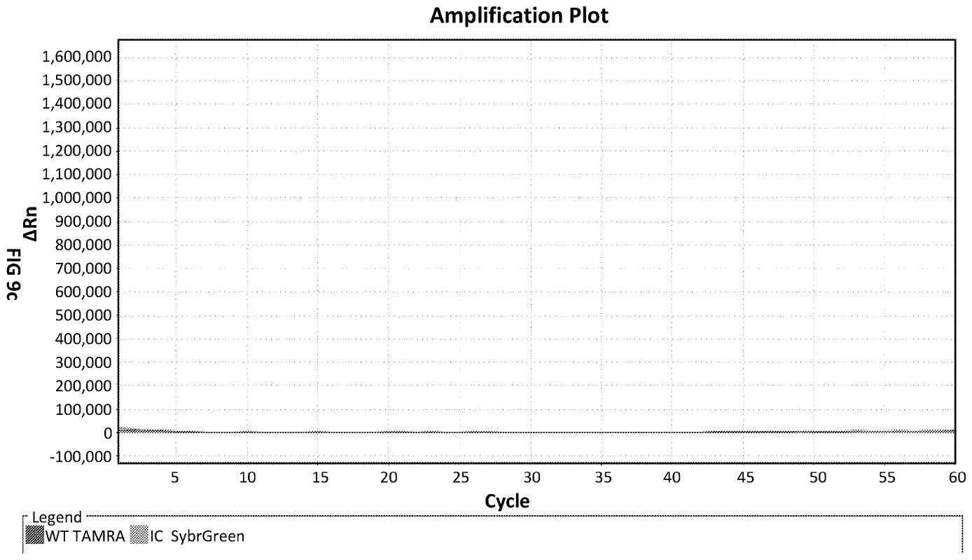

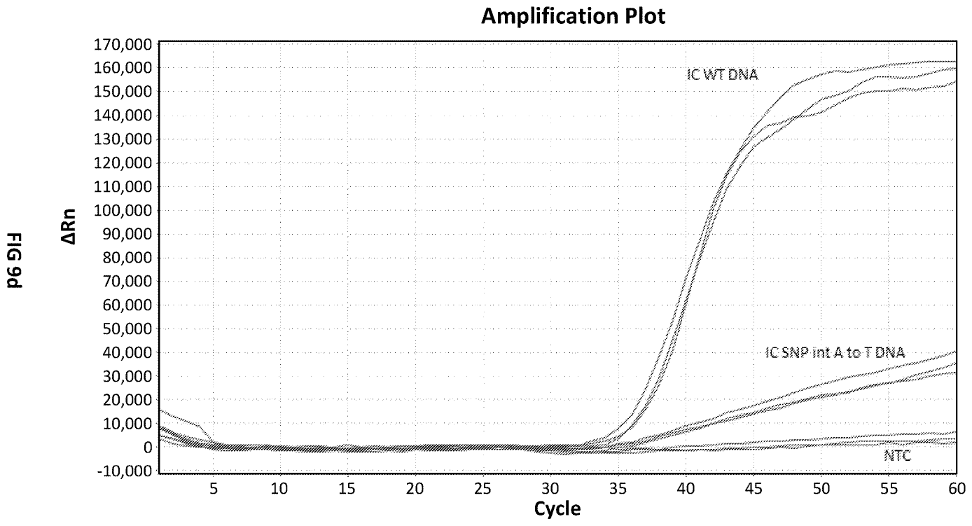

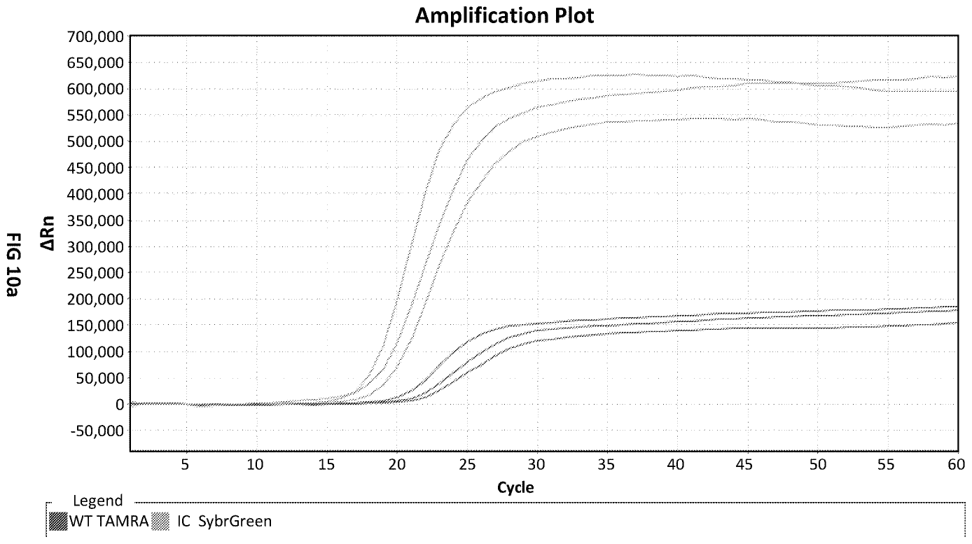

FIGS. 9a to 9d show LAMP experiments conducted according to a method of the present invention in the presence of a fluorescent WT probe, an intercalating dye and a SNP internal C to T blank oligo. As shown in FIG. 9a, in the presence of a WT sequence in a sample, the fluorescence both the probe and the intercalating due displayed an increase in signal as the LAMP reaction progressed, because both fluorescent components were able to bind to the amplified DNA. However, as shown in FIG. 9b, in the presence of a sample which only comprises a SNP internal C to T (which differs from the WT sequence by a single nucleotide), only the intercalating dye showed any significant increase in signal. Thus, the combination of a probe and blank oligo having a single-point mutation in the middle of the sequence was able to distinguish and detect WT and SNP-containing sequences with high specificity in the CHES CAPSO buffer environment. FIG. 9c is an NTC control experiment and showed no significant increase in fluorescence. FIG. 9d shows a comparison of output signals in the TAMRA channel in the presence of WT or SNP containing sequences.

TABLE 9
results for SNP internal C to T under
CHES CAPSO Buffer conditions.

| | | SNP int | No template |
|---|---|---|---|
| | WT DNA | C to T DNA | control (H₂O) |
| WT - TAMRA | Detected | Not detected | Not detected |
| SybrGreen control | Detected | Detected | Not detected |

Experiment 3d—CHES CAPSO Buffer (SNP Internal A to T)

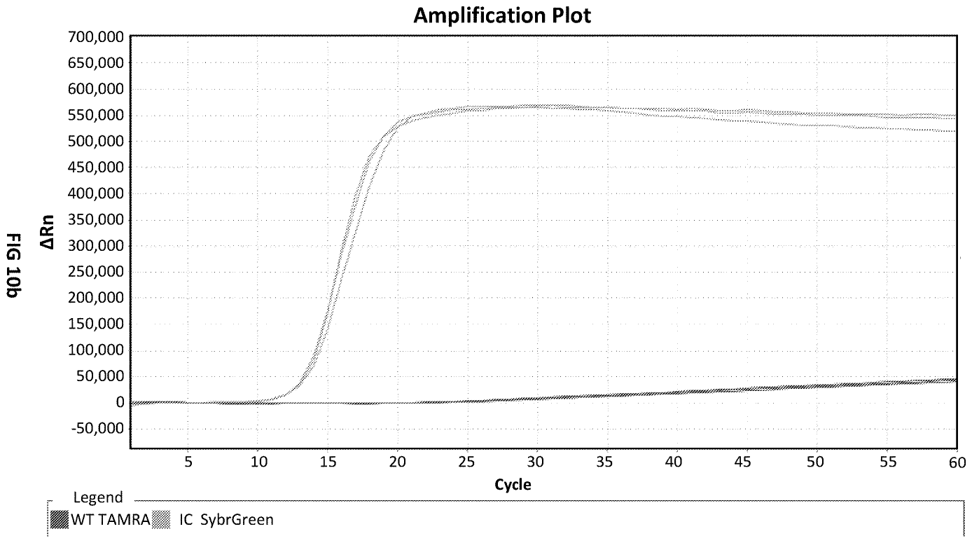

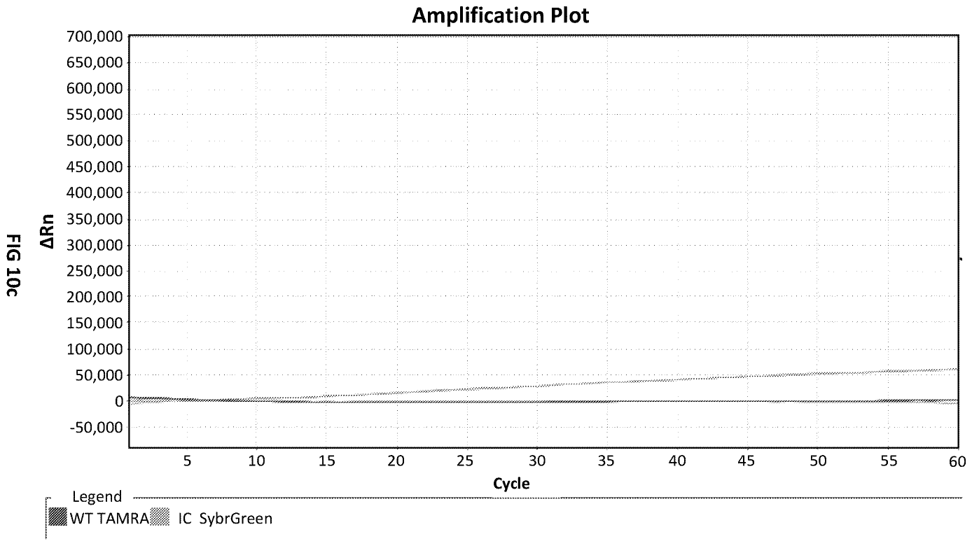

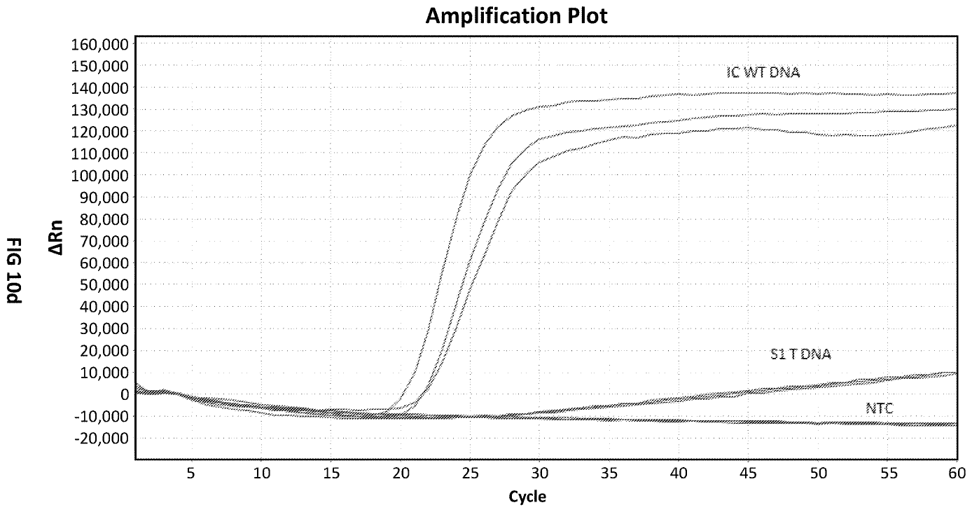

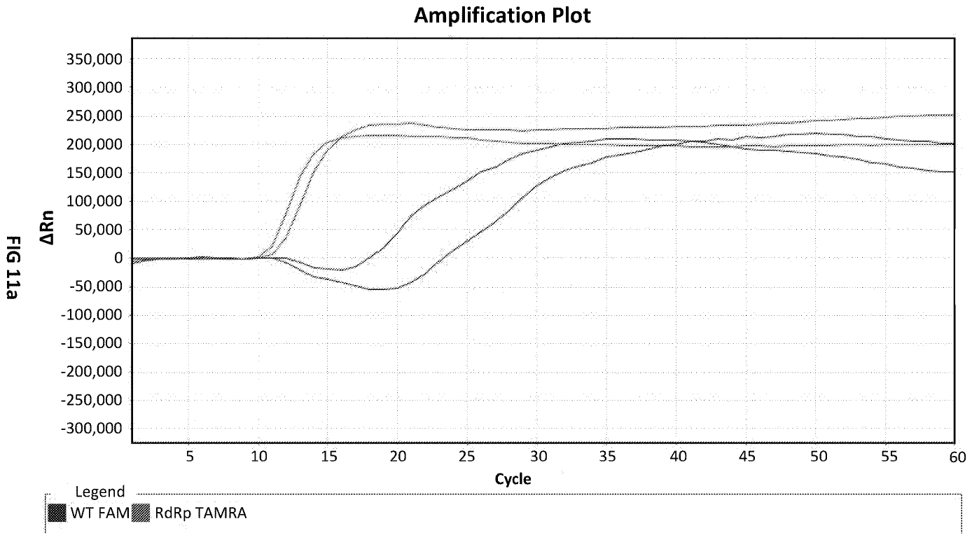

Experiment 3b was repeated with a SNP internal A to T sample, WT-TAMRA probe and a SNP internal A to T blank oligo using CHES CAPSO instead of RB (Tris) buffer. FIGS. 10a to 10d show plots of fluorescent signal from LAMP amplification conducted according to a method of the invention in the presence of a fluorescent WT probe, an intercalating dye and a SNP internal A to T blank oligo. As shown in FIG. 10a, in the presence of a WT sample sequence, the fluorescent signal of both the probe and the intercalating dye increased as the LAMP reaction progressed, indicating that both fluorescent components were able to bind to the amplified DNA.

When the amplification was carried out on the mutant SNP internal A to T sample, as shown in FIG. 10b, only the intercalating dye showed any significant increase in signal. Thus, change of buffer from the RB (tris) to CHES CAPSO increased the specificity of the assay when the SNP was located on the middle portion of the blank oligo. FIG. 7c shows a control NTC reaction that showed no signal. FIG. 10d shows a comparison of output signals in the TAMRA channel in the presence of WT or SNP containing sequences.

The amplification times obtained using the CHES CAPSO buffer were slower than those obtained using the RB buffer. However, these experiments demonstrated that in the CHES CAPSO buffer environment it is possible to detect point mutations located in the middle of the blank oligo sequence with high specificity. This approach may therefore provide a viable means to detect SNPs located in the middle of blank oligo sequences, especially where abundant amounts of DNA are present in the sample (for example in confirmatory tests from culture).

### Sequences:

C-position of the fluorophore
A-SNP mutation at or near 3′ end
SEQ ID NO. 22: WT sample:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

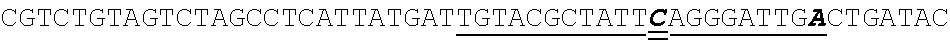

CGGAAGACATCTCAAATGAAGTGGTCTATGCGACAGAGAC
SEQ ID NO. 23: WT probe:
TGTACGCTATTCAGGGATTGA

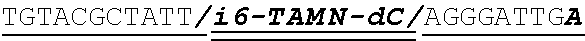

SEQ ID NO. 24: SNP internal C to T sample:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA
CGTCTGTAGTCTAGCCTCATTATGATTGTACGCTATTTAGGGATTGACTGATACC
GGAAGACATCTCAAATGAAGTGGTCTATGCGACAGAGAC
SEQ ID NO. 25: SNP internal C to T blank oligo:
TGTACGCTATTTAGGGATTGA
SEQ ID NO. 26: IC SNP internal A to T sample:
AGCCCACGGAGACCACTGACCGATCTACCTGAACGGCGACCATCTGTGTGGTA
CTGGGGCGGAGAGATAACTACGGTGCCGCTTACAGCCCCTCTGTCGTCGCTGA

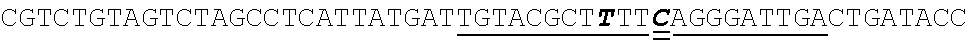

GGAAGACATCTCAAATGAAGTGGTCTATGCGACAGAGAC
SEQ ID NO. 27: SNP internal A to T blank oligo:
TGTACGCTTTTCAGGGATTGA

### Example 4

Examples 1-3 using probes and artificial DNA sequences with a point mutation show that detection of SNPs with LAMP is achievable using competitive probes and/or blank oligos covering the sequence with the point mutation and the SNP located at 3′ end or middle portion of each probe (one WT probe and one mutant SNP probe or blank oligo).

However, SNPs can also be detected without the use of a competitive probe or blank oligo, provided that the probe produces a sufficiently specific signal on its own.

### Method

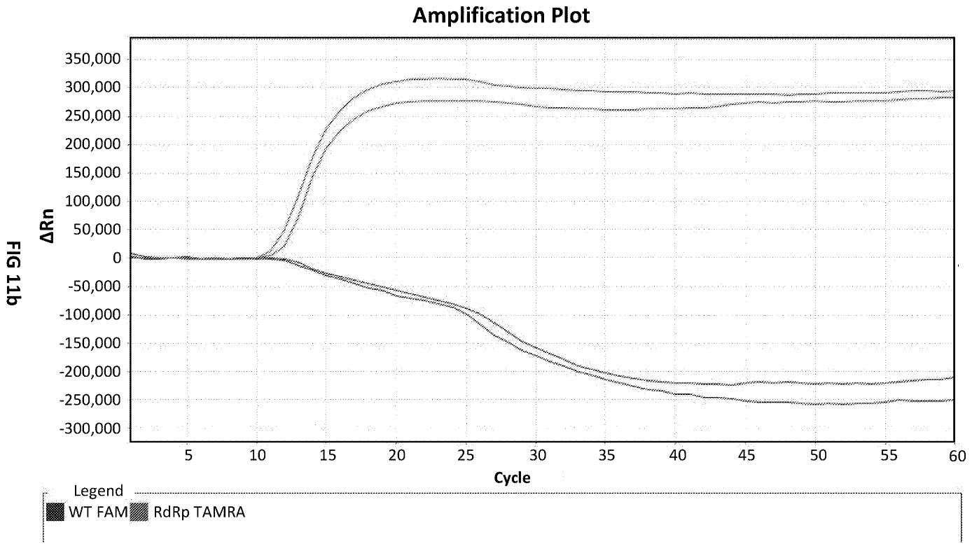

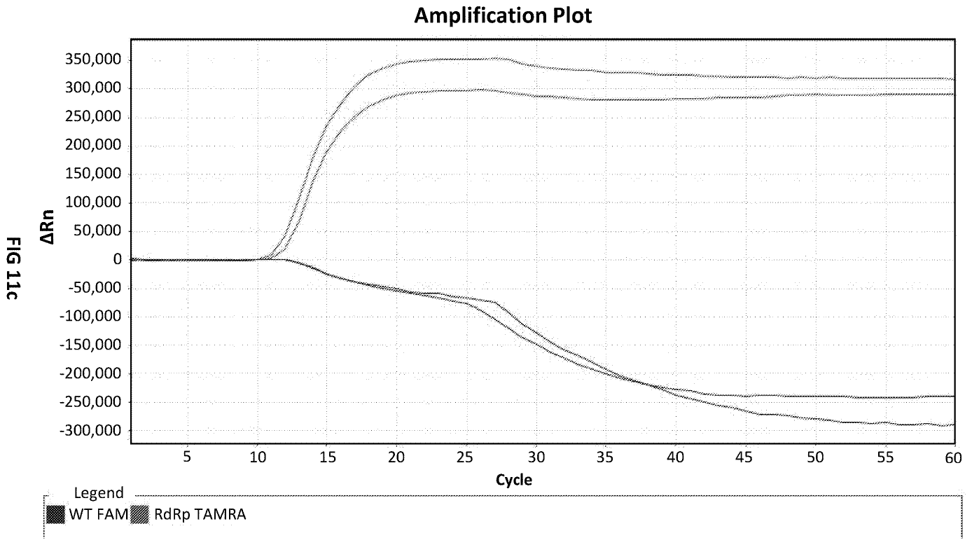

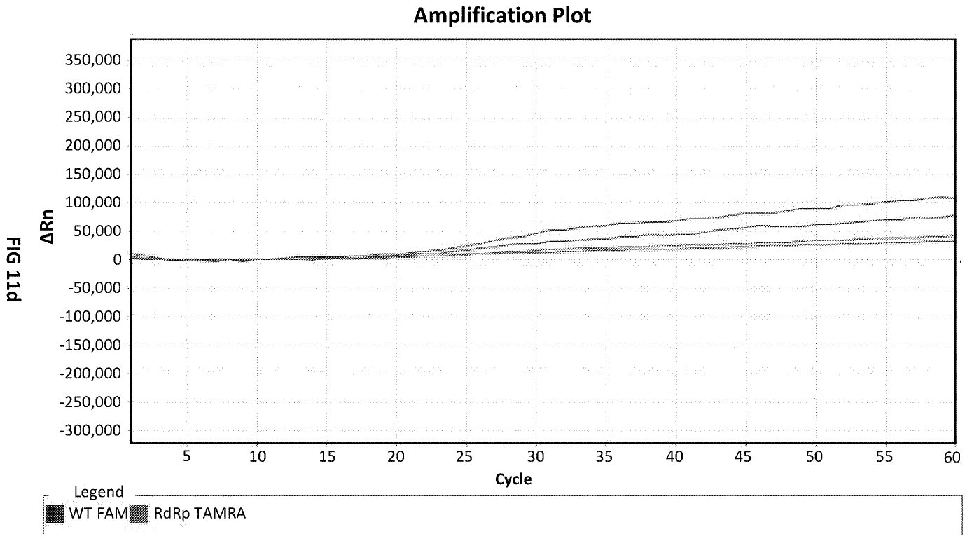

This method was used to detect the A23063T (N501Y) mutation in B 1.1.7 SARS-CoV-2 variant. A23063T (N501Y) mutation occurs in the S gene of B 1.1.7 variant of SARA-COV-2 virus but not in the wild type virus. In the described assay two set of DNA primers and probes were used: an S gene primer set, covering the region with A23063T (N501Y) mutation and RdRp gene, which does not hold any mutations in the B 1.1.7 variant. The assay also included two probes labelled with two different fluorophores: WT-FAM probe compatible with the S gene primer set and RdRp-TAMRA probe compatible with the RdRp primer set. The WT-FAM probe was based on wild type SARS-CoV-2 sequence (without the mutation), however it was designed in such a way that the 3′end of one of the loop primers was located at the position where the A23063T (N501Y) occurs in the B 1.1.7 SARS-CoV-2 variant whereas the RdRp-TAMRA probe was used as a generic probe to generate a fluorescent signal both with wild type and B 1.1.7 variant. The tests were performed using 10×RT pellets containing BST polymerase and RTX reverse transcriptase and 5×MELT buffer (0.01M tris buffer pH 8 with 0.1M Tween 20). WT-FAM probe (for S gene) and RdRp-TAMRA probe were tested in conjunction at the same concentrations: 2.4 μl of 10 UM probe per 1 10×RT pellet with 1000 copiers per reaction of WT (FIG. 11a) or B 1.1.7 RNA from two different suppliers-ATCC (FIG. 11b) or VIRCELL (FIG. 11c) or with molecular grade water—NTC (FIG. 11d). No blank oligos were used in this assay set up.

### Results

The generic RdRp-TAMRA control probe generated fluorescence signal in the presence of all tested RNA samples (FIG. 11a, b and c) but not in the presence of no template controls (FIG. 11d), whereas the WT-FAM probe generated fluorescent signal only in the presence of WT SARS-CoV-2 RNA (FIG. 11a) but not RNA samples from the B 1.1.7 variant (FIGS. 11b and c).

These results demonstrate that SNP can be detected also in the absence of blank oligo or a competing probe providing the first probe generates sufficiently specific signal.

### Sequences:

```
C - position of the fluorophore

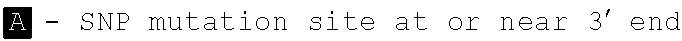

SEQ ID NO. 28: WT S gene sequence:
21541 CTTGTTAACAACTAAACGAACAATGTTTGTTTTTCTTGTTTTATTGCCACTAGTCTCTAG
21601 TCAGTGTGTTAATCTTACAACCAGAACTCAATTACCCCCTGCATACACTAATTCTTTCAC
21661 ACGTGGTGTTTATTACCCTGACAAAGTTTTCAGATCCTCAGTTTTACATTCAACTCAGGA
21721 CTTGTTCTTACCTTTCTTTTCCAATGTTACTTGGTTCCATGCTATACATGTCTCTGGGAC
21781 CAATGGTACTAAGAGGTTTGATAACCCTGTCCTACCATTTAATGATGGTGTTTATTTTGC
21841 TTCCACTGAGAAGTCTAACATAATAAGAGGCTGGATTTTTGGTACTACTTTAGATTCGAA
21901 GACCCAGTCCCTACTTATTGTTAATAACGCTACTAATGTTGTTATTAAAGTCTGTGAATT
21961 TCAATTTTGTAATGATCCATTTTTGGGTGTTTATTACCACAAAAACAACAAAAGTTGGAT
22021 GGAAAGTGAGTTCAGAGTTTATTCTAGTGCGAATAATTGCACTTTTGAATATGTCTCTCA
22081 GCCTTTTCTTATGGACCTTGAAGGAAAACAGGGTAATTTCAAAAATCTTAGGGAATTTGT
22141 GTTTAAGAATATTGATGGTTATTTTAAAATATATTCTAAGCACACGCCTATTAATTTAGT
22201 GCGTGATCTCCCTCAGGGTTTTTCGGCTTTAGAACCATTGGTAGATTTGCCAATAGGTAT
22261 TAACATCACTAGGTTTCAAACTTTACTTGCTTTACATAGAAGTTATTTGACTCCTGGTGA
22321 TTCTTCTTCAGGTTGGACAGCTGGTGCTGCAGCTTATTATGTGGGTTATCTTCAACCTAG
22381 GACTTTTCTATTAAAATATAATGAAAATGGAACCATTACAGATGCTGTAGACTGTGCACT
22441 TGACCCTCTCTCAGAAACAAAGTGTACGTTGAAATCCTTCACTGTAGAAAAAGGAATCTA
22501 TCAAACTTCTAACTTTAGAGTCCAACCAACAGAATCTATTGTTAGATTTCCTAATATTAC
22561 AAACTTGTGCCCTTTTGGTGAAGTTTTTAACGCCACCAGATTTGCATCTGTTTATGCTTG
22621 GAACAGGAAGAGAATCAGCAACTGTGTTGCTGATTATTCTGTCCTATATAATTCCGCATC
22681 ATTTTCCACTTTTAAGTGTTATGGAGTGTCTCCTACTAAATTAAATGATCTCTGCTTTAC
22741 TAATGTCTATGCAGATTCATTTGTAATTAGAGGTGATGAAGTCAGACAAATCGCTCCAGG
22801 GCAAACTGGAAAGATTGCTGATTATAATTATAAATTACCAGATGATTTTACAGGCTGCGT
22861 TATAGCTTGGAATTCTAACAATCTTGATTCTAAGGTTGGTGGTAATTATAATTACCTGTA
22921 TAGATTGTTTAGGAAGTCTAATCTCAAACCTTTTGAGAGAGATATTTCAACTGAAATCTA
22981 TCAGGCCGGTAGCACACCTTGTAATGGTGTTGAAGGTTTTAATTGTTACTTTCCTTTACA

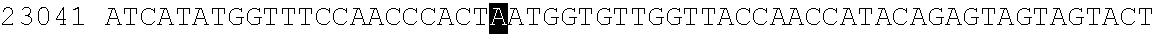

23101 TTCTTTTGAACTTCTACATGCACCAGCAACTGTTTGTGGACCTAAAAAGTCTACTAATTT
23161 GGTTAAAAACAAATGTGTCAATTTCAACTTCAATGGTTTAACAGGCACAGGTGTTCTTAC
23221 TGAGTCTAACAAAAAGTTTCTGCCTTTCCAACAATTTGGCAGAGACATTGCTGACACTAC
23281 TGATGCTGTCCGTGATCCACAGACACTTGAGATTCTTGACATTACACCATGTTCTTTTGG
23341 TGGTGTCAGTGTTATAACACCAGGAACAAATACTTCTAACCAGGTTGCTGTTCTTTATCA
23401 GGATGTTAACTGCACAGAAGTCCCTGTTGCTATTCATGCAGATCAACTTACTCCTACTTG
23461 GCGTGTTTATTCTACAGGTTCTAATGTTTTTCAAACACGTGCAGGCTGTTTAATAGGGGC
23521 TGAACATGTCAACAACTCATATGAGTGTGACATACCCATTGGTGCAGGTATATGCGCTAG
23581 TTATCAGACTCAGACTAATTCTCCTCGGCGGGCACGTAGTGTAGCTAGTCAATCCATCAT
23641 TGCCTACACTATGTCACTTGGTGCAGAAAATTCAGTTGCTTACTCTAATAACTCTATTGC
23701 CATACCCACAAATTTTACTATTAGTGTTACCACAGAAATTCTACCAGTGTCTATGACCAA
23761 GACATCAGTAGATTGTACAATGTACATTTGTGGTGATTCAACTGAATGCAGCAATCTTTT
23821 GTTGCAATATGGCAGTTTTTGTACACAATTAAACCGTGCTTTAACTGGAATAGCTGTTGA
23881 ACAAGACAAAAACACCCAAGAAGTTTTTGCACAAGTCAAACAAATTTACAAAACACCACC
23941 AATTAAAGATTTTGGTGGTTTTAATTTTTCACAAATATTACCAGATCCATCAAAACCAAG
24001 CAAGAGGTCATTTATTGAAGATCTACTTTTCAACAAAGTGACACTTGCAGATGCTGGCTT
24061 CATCAAACAATATGGTGATTGCCTTGGTGATATTGCTGCTAGAGACCTCATTTGTGCACA
24121 AAAGTTTAACGGCCTTACTGTTTTGCCACCTTTGCTCACAGATGAAATGATTGCTCAATA
24181 CACTTCTGCACTGTTAGCGGGTACAATCACTTCTGGTTGGACCTTTGGTGCAGGTGCTGC
24241 ATTACAAATACCATTTGCTATGCAAATGGCTTATAGGTTTAATGGTATTGGAGTTACACA
24301 GAATGTTCTCTATGAGAACCAAAAATTGATTGCCAACCAATTTAATAGTGCTATTGGCAA
24361 AATTCAAGACTCACTTTCTTCCACAGCAAGTGCACTTGGAAAACTTCAAGATGTGGTCAA
24421 CCAAAATGCACAAGCTTTAAACACGCTTGTTAAACAACTTAGCTCCAATTTTGGTGCAAT
24481 TTCAAGTGTTTTAAATGATATCCTTTCACGTCTTGACAAAGTTGAGGCTGAAGTGCAAAT
24541 TGATAGGTTGATCACAGGCAGACTTCAAAGTTTGCAGACATATGTGACTCAACAATTAAT
24601 TAGAGCTGCAGAAATCAGAGCTTCTGCTAATCTTGCTGCTACTAAAATGTCAGAGTGTGT
24661 ACTTGGACAATCAAAAAGAGTTGATTTTTGTGGAAAGGGCTATCATCTTATGTCCTTCCC
24721 TCAGTCAGCACCTCATGGTGTAGTCTTCTTGCATGTGACTTATGTCCCTGCACAAGAAAA
24781 GAACTTCACAACTGCTCCTGCCATTTGTCATGATGGAAAAGCACACTTTCCTCGTGAAGG
24841 TGTCTTTGTTTCAAATGGCACACACTGGTTTGTAACACAAAGGAATTTTTATGAACCACA
24901 AATCATTACTACAGACAACACATTTGTGTCTGGTAACTGTGATGTTGTAATAGGAATTGT
24961 CAACAACACAGTTTATGATCCTTTGCAACCTGAATTAGACTCATTCAAGGAGGAGTTAGA
25021 TAAATATTTTAAGAATCATACATCACCAGATGTTGATTTAGGTGACATCTCTGGCATTAA
25081 TGCTTCAGTTGTAAACATTCAAAAAGAAATTGACCGCCTCAATGAGGTTGCCAAGAATTT
25141 AAATGAATCTCTCATCGATCTCCAAGAACTTGGAAAGTATGAGCAGTATATAAAATGGCC
25201 ATGGTACATTTGGCTAGGTTTTATAGCTGGCTTGATTGCCATAGTAATGGTGACAATTAT
25261 GCTTTGCTGTATGACCAGTTGCTGTAGTTGTCTCAAGGGCTGTTGTTCTTGTGGATCCTG
25321 CTGCAAATTTGATGAAGACGACTCTGAGCCAGTGCTCAAAGGAGTCAAATTACATTACAC
WT-FAM Probe design:
Reverse complement
                                                                                                      (SEQ ID NO. 29)

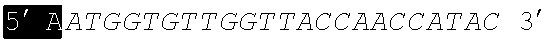

                                                                                                      (SEQ ID NO. 30)
5' GTATGGTTGGTAACCAACACCATT 3'
WT-FAM Probe:
                                                                                                      (SEQ ID NO. 31)

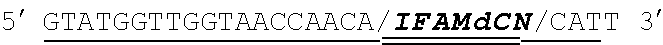

RdRp-TAMRA Probe:
                                                                                                      (SEQ ID NO. 32)

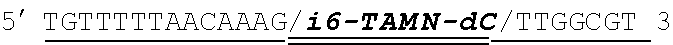

```

### Primer Sequences:

S gene primer sequences compatible with WT-FAM probe

| | |
|---|---|
| SEQ ID NO. 33: S set 4 | CTGTATAGATTGTTTAGGAAGTCT |
| F3 | |
| SEQ ID NO. 34: S set 4 | GCTGGTGCATGTAGAAGT |
| B3 | |
| SEQ ID NO. 35: S set 4 | TCAACACCATTACAAGGTGTGCT |
| FIP | AAATCTCAAACCTTTTGAGAGAG |
| SEQ ID NO. 36: S set 4 | TACAATCATATGGTTTCCAACCC |
| BIP | ACTCAAAAGAAAGTACTACTACT |
| | CTGT |
| SEQ ID NO. 37: S set 4 | CGGCCTGATAGATTTCAGTTGAAA |
| LF | |

RdRp primer sequences compatible with RdRp-TAMRA probe

| | |
|---|---|
| SEQ ID NO. 38: nCoVr-ID01- | AAAACCCAGATATATTACGCG |
| F3 | |
| SEQ ID NO. 39: nCoVr-ID01- | CTACAACAGGAACTCCACT |
| B3 | |
| SEQ ID NO. 40: nCoVr-ID01- | CGCATGGCATCACAGAATTGT |
| FIP | CGCCAACTTAGGTGAACG |
| SEQ ID NO. 41: nCoVr-ID01- | TGCTGGTATTGTTGGTGTACT |
| BIP | GGGTTTGTATGAAATCACCGAA |
| SEQ ID NO. 42: nCoVr-ID01- | TGTTTTTAACAAAGCTTGGCGT |
| LF | |
| SEQ ID NO. 43: nCoVr-ID01- | TCAAGATCTCAATGGTAACTGGT |
| LB | |

### CLAUSES

1. A method for detecting single nucleotide polymorphisms (SNPs) in a target nucleic acid sequence in a sample, the method comprising:
   a) providing a first oligonucleotide probe complementary to a wild-type target sequence, wherein the first oligonucleotide probe comprises a fluorescent label bound to an internal cytosine base and wherein said oligonucleotide probe does not have a 3′ end terminator;
   b) amplifying a target nucleic acid sequence in the sample to provide an amplified nucleic acid by a loop-mediated isothermal amplification in a reaction vessel with the first oligonucleotide probe and a second probe or intercalating dye, and probing the amplified nucleic acid sequence;
   c) detecting the presence of the target wild-type and/or a mutant nucleic acid sequence; and
   d) distinguishing between the presence of the target wild-type and/or mutant nucleic acid sequence based on the fluorescent labeling.
2. The method of clause 1, further comprising:
   providing an intercalating dye or providing a second probe, wherein the second probe is selected from a probe complementary to a mutant sequence of the wild-type target sequence or a generic probe capable of detecting both the wild-type target sequence and the mutant sequence of the wild-type target sequence;
   wherein the second probe and/or intercalating dye comprise a further fluorescent label distinguishable from the first probe; and
   wherein the target nucleic acid sequence is amplified in the reaction vessel with the second probe or intercalating dye.
3. The method of any one of clauses 1 and 2, wherein at least one of the probes comprises a single- or double-point mutation.
4. The method of any preceding clause, wherein the method provides the second probe, wherein the second probe is complementary to a mutant sequence of the wild-type target sequence, and:
   i) further comprises a fluorescent label bound to an internal cytosine base and does not have a 3′ end terminator; and
   ii) wherein at least one of the first and second probes contains a single- or double-point mutation at or near the 3′ end.
5. The method of any clauses 1-3, wherein the method provides the intercalating dye or a generic probe, and wherein:
   a) the method further provides a blank oligo having the same sequence as the first probe but absent a fluorescent label, and comprising one or more SNP mutations at or near 3′ end; or
   b) the method further provides a blank oligo having the same sequence as the first probe but absent a fluorescent label, and comprising one or more SNPs in the middle of the sequence, optionally wherein the method uses a CHES CAPSO buffer; or
   c) wherein the method omits a blank oligo, and wherein the first probe is specific to wild-type DNA or RNA sequence having a single point mutation positioned opposite the nucleotide located at or near the 3′ end of the probe.
6. The method of any preceding clause, wherein the fluorescent labels and/or intercalating dye comprise one or more of the following: TAMRA, FAM, Cy5, SYBR Green, EvaGreen, JOE, TET, HEX, ROX, ALEXA, ATTO and v13, or any other dye suitable for nucleic acid detection.
7. The method of any preceding clause, wherein:
   a) the first probe and/or second probe is configured to function as a loop-mediated isothermal amplification primer, optionally an LF or LB loop primer; and/or
   b) the first probe, the second probe and/or the target nucleic acid sequence is:
      i) a DNA sequence; or
      ii) an RNA sequence.
8. The method of any preceding clause, wherein the loop-mediated isothermal amplification is carried out using an RB (tris) buffer.
9. The method of any preceding clause, wherein the first or second probe comprises one or more of the following sequences:

SEQ ID NO. 1:
5′ GTATGGTTGGTAACCAACA/IFAMdCN/CATT 3′
(SARS-CoV-2 WT probe S gene for distinction
of A23063T N501Y point mutation);
SEQ ID NO. 2:
5′ AAAGGAAAGTAACAATTAAAAC/i6-TAMN-dC/TTC 3′
(SARS-CoV-2 WT probe S gene for distinction
of E484K point mutation);
or
SEQ ID NO. 3:
5′ AAAGGAAAGTAACAATTAAAACCTTT 3′
(SARS-CoV-2 mutant blank oligo for
distinction of E484K point mutation).

10. The method of any preceding clause, wherein the presence of the target wild-type and/or mutant nucleic acid sequence can be detected:
   i) during loop-mediated isothermal amplification, and/or
   ii) without any post-amplification manipulation.
11. The method of any preceding clause, wherein the target nucleic acid is from a virus, optionally wherein the virus is SARS-CoV-2 or a variant thereof.
12. The method of any preceding clause, wherein the loop-mediated isothermal amplification uses FIP, BIP, F3 and B3 primers, optionally wherein the amplification also uses loop F and/or Loop B primers.
13. The method of any preceding clause, wherein the LAMP reaction and the detection of the amplified nucleic acid sequences are carried out in the same vessel
14. A kit for detecting nucleotide polymorphisms in a target nucleic acid sequence in a sample, the kit comprising:
   a) a first oligonucleotide probe complementary to a wild-type target sequence, wherein the first oligonucleotide probe comprises a fluorescent label bound to an internal cytosine base and wherein said oligonucleotide probe sequence does not have a 3′ end terminator; and
   b) a loop-mediated isothermal amplification reagent buffer, enzyme, dNTPs and loop-mediated isothermal amplification primers; and
      wherein the first probe is configured to be used in a single reaction vessel.
15. The kit of clause 14, further comprising:
   an intercalating dye or a second oligonucleotide probe, wherein the second oligonucleotide probe is selected from a probe complementary to a mutant sequence of the wild-type target sequence, or a generic probe capable of detecting both the wild-type and mutant target sequence;
   wherein the second oligonucleotide probe and/or intercalating dye comprise a further fluorescent label distinguishable from the first probe; and
   the first probe, the second probe and/or the intercalating dye are configured to be used in a single reaction vessel.
16. The kit of any one of clauses 14 and 15, wherein at least one of the probes comprises a single- or double-point mutation.
17. The kit of any of clauses 15 and 16, the kit providing the second probe, wherein the second probe is complementary to a mutant sequence of the wild-type target sequence and:
   i) further comprises a fluorescent label bound to an internal cytosine base and does not have a 3′ end terminator; and
   ii) wherein at least one of the first and second probes contains a single- or double-point mutation at or near the 3′ end.
18. The kit of any of clauses 15 or 16, wherein the kit provides the intercalating dye or a generic probe, and wherein:
   a) the kit further comprises a blank oligo having the same sequence as the first probe but absent a fluorescent label, and comprising one or more SNP mutations at or near 3′ end; or
   b) the kit further comprises s a blank oligo having the same sequence as the first probe but absent a fluorescent label, and comprising one or more SNP in the middle of the sequence, optionally wherein the method uses a CHES CAPSO buffer; or
   c) wherein the kit omits a blank oligo, and wherein the first probe is specific to wild-type DNA or RNA sequence having a single point mutation positioned opposite the nucleotide located at or near the 3′ end of the probe.
19. The kit of any of clauses 14-18, wherein the fluorescent labels and/or intercalating dye comprise one or more of the following: TAMRA, FAM, Cy5, SYBR Green, EvaGreen, JOE, TET, HEX, ROX, ALEXA, ATTO and v13, or any other dye suitable for nucleic acid detection.
20. The kit of any of clauses 14-19, wherein:
   a) first the probe and/or second probe is configured to function as a loop-mediated isothermal amplification primer; and/or
   b) the first probe, the second probe and/or the target nucleic acid sequence is:
      i) a DNA sequence; or
      ii) an RNA sequence.
21. The kit of any of clauses 14-20, wherein the buffer is an RB (tris) buffer.
22. The kit of any of clauses 14-21, wherein the first or second probe comprises one or more of the following sequences:

SEQ ID NO. 1:
5′ GTATGGTTGGTAACCAACA/IFAMdCN/CATT 3′
(SARS-CoV-2 WT probe S gene for distinction
of A23063T N501Y point mutation),
SEQ ID NO. 2:
5′ AAAGGAAAGTAACAATTAAAAC/i6-TAMN-dC/TTC 3′
(SARS-CoV-2 WT probe S gene for distinction
of E484K point mutation),
and
SEQ ID NO. 3:
5′ AAAGGAAAGTAACAATTAAAACCTTT 3′
(SARS-CoV-2 mutant blank oligo
for distinction of E484K point mutation).

23. The kit of any of clauses 14-22, wherein the kit is configured to allow detection of the presence of the target wild-type and/or mutant nucleic acid sequence:
   i) during loop-mediated isothermal amplification, and/or
   ii) without any post-amplification manipulation.
24. The kit of any of clauses 14-23, wherein the target nucleic acid is from a virus, optionally where in the virus SARS-CoV-2 or a variant thereof.
25. The kit of any of clauses 14-24, wherein the loop-mediated isothermal amplification uses FIP, BIP, F3 and B3 primers, optionally wherein the amplification also uses loop F and/or Loop B primers.

1. A method for detecting and/or distinguishing single nucleotide polymorphisms (SNPs) in a target nucleic acid sequence in a sample, the method comprising:
a) providing a first oligonucleotide probe complementary to a wild-type target sequence, wherein the first oligonucleotide probe comprises a fluorescent label bound to an internal cytosine base and wherein said oligonucleotide probe does not have a 3′ end terminator;
b) providing a second oligonucleotide probe comprising a further fluorescent label distinguishable from the first probe or a blank oligo that is absent a fluorescent label, wherein the second oligonucleotide probe or blank oligo is complementary to a mutant sequence of the wild-type target sequence, and containing a single or double point mutation which distinguishes the mutation;
c) amplifying a target nucleic acid sequence in the sample to provide an amplified nucleic acid by a loop-mediated isothermal amplification in a reaction vessel with the first oligonucleotide probe and:
i) the second oligonucleotide probe, or
ii) the blank oligo and an intercalating dye or generic probe,
and probing the amplified nucleic acid sequence;
d) detecting the presence of the target wild-type and/or a mutant nucleic acid sequence; and
e) distinguishing between the presence of the target wild-type and/or mutant nucleic acid sequence based on the fluorescent labeling.

2. The method of claim 1, wherein:
the generic probe is capable of detecting both the wild-type target sequence and the mutant sequence of the wild-type target sequence;
wherein the generic probe or intercalating dye comprise a further fluorescent label distinguishable from the first probe; and
wherein the target nucleic acid sequence is amplified in the reaction vessel with the generic probe or intercalating dye.

3. The method of claim 1, wherein the method provides the second probe, and:
i) the second probe further comprises a fluorescent label bound to an internal cytosine base and does not have a 3′ end terminator; and
ii) wherein the first and second probes contain a single- or double-point mutation at or near the 3′ end.

4. The method of claim 1, wherein the method provides the intercalating dye or the generic probe, and wherein:
a) the blank oligo comprises one or more SNP mutations at or near 3′ end; or
b) the blank oligo comprises one or more SNP mutations in the middle of the sequence, optionally wherein the method uses a CHES CAPSO buffer.

5. The method of claim 1, wherein the fluorescent labels and/or intercalating dye comprise one or more of the following: TAMRA, FAM, Cy5, SYBR Green, EvaGreen, JOE, TET, HEX, ROX, ALEXA, ATTO and v13, or any other dye suitable for nucleic acid detection.

6. The method of claim 1, wherein:
a) the first probe and/or second probe or blank oligo is configured to function as a loop-mediated isothermal amplification primer, optionally an LF or LB loop primer; and/or
b) the first probe, the second probe, blank oligo and/or the target nucleic acid sequence is:
i) a DNA sequence; or
ii) an RNA sequence.

7. The method of claim 1, wherein the loop-mediated isothermal amplification is carried out using an RB (tris) buffer.

8. The method of claim 1, wherein the first or second probe comprises one or more of the following sequences:
SEQ ID NO. 1:
5′ GTATGGTTGGTAACCAACA/IFAMdCN/CATT 3′
(SARS-CoV-2 WT probe S gene for distinction
of A23063T N501Y point mutation);
SEQ ID NO. 2:
5′ AAAGGAAAGTAACAATTAAAAC/16-TAMN-dC/TTC 3′
(SARS-CoV-2 WT probe S gene for distinction
of E484K point mutation);
or
SEQ ID NO. 3:
5′ AAAGGAAAGTAACAATTAAAACCTTT 3′
(SARS-CoV-2 mutant blank oligo for
distinction of E484K point mutation).

9. The method of claim 1, wherein the presence of the target wild-type and/or mutant nucleic acid sequence can be detected:
i) during loop-mediated isothermal amplification, and/or
ii) without any post-amplification manipulation.

10. The method of claim 1, wherein the target nucleic acid is from a virus, optionally wherein the virus is SARS-CoV-2 or a variant thereof.

11. The method of claim 1, wherein the loop-mediated isothermal amplification uses FIP, BIP, F3 and B3 primers, optionally wherein the amplification also uses loop F and/or Loop B primers.

12. The method of claim 1, wherein the LAMP reaction and the detection of the amplified nucleic acid sequences are carried out in the same vessel.

13. The method of claim 1, wherein the blank oligo is provided at a concentration that is equal to or in excess of the concentration of the first oligonucleotide probe.

14. The method of claim 13, wherein the blank oligo is provided at a concentration of two or more times the concentration of the first oligonucleotide probe, optionally wherein the blank oligo is provided at a concentration of four or more times the concentration of the first oligonucleotide probe.

15. A kit for distinguishing nucleotide polymorphisms in a target nucleic acid sequence in a sample, the kit comprising:
a) a first oligonucleotide probe complementary to a wild-type target sequence, wherein the first oligonucleotide probe comprises a fluorescent label bound to an internal cytosine base and wherein said oligonucleotide probe sequence does not have a 3′ end terminator; and
i) a second oligonucleotide probe comprising a further fluorescent label distinguishable from the first probe, wherein the second oligonucleotide probe is complementary to a mutant sequence of the wild-type target sequence, and containing a single or double point mutation at or near the 3′ end which distinguishes the mutation; or
ii) a blank oligo complementary to a mutant sequence of the wild-type target sequence, and containing a single or double point mutation which distinguishes the mutation, and an intercalating dye or generic probe; and
b) a loop-mediated isothermal amplification reagent buffer, enzyme, dNTPs and loop-mediated isothermal amplification primers; and
wherein the first probe and second probe or blank oligo is configured to be used in a single reaction vessel.

16. The kit of claim 15, wherein:
the generic probe is capable of detecting both the wild-type and mutant target sequence;
wherein the generic probe or intercalating dye comprise a further fluorescent label distinguishable from the first probe; and
the first probe and the generic probe or the intercalating dye are configured to be used in a single reaction vessel.

17. The kit of claim 15, the kit providing the second probe, and:
i) the second probe further comprises a fluorescent label bound to an internal cytosine base and does not have a 3′ end terminator; and
ii) wherein the first probe contains a single- or double-point mutation at or near the 3′ end.

18. The kit of claim 16, wherein the kit provides the intercalating dye or the generic probe, and wherein:
a) the blank oligo comprises one or more SNP mutations at or near 3′ end; or
b) the blank oligo comprises one or more SNP mutations in the middle of the sequence, optionally wherein the method uses a CHES CAPSO buffer.

19. The kit of claim 15, wherein the fluorescent labels and/or intercalating dye comprise one or more of the following: TAMRA, FAM, Cy5, SYBR Green, EvaGreen, JOE, TET, HEX, ROX, ALEXA, ATTO and v13, or any other dye suitable for nucleic acid detection.

20. The kit of claim 15, wherein:
a) the first probe, blank oligo and/or second probe is configured to function as a loop-mediated isothermal amplification primer, optionally wherein the; and/or
b) the first probe, the blank oligo, the second probe and/or the target nucleic acid sequence is:
i) a DNA sequence; or
ii) an RNA sequence.

21. The kit of claim 15, wherein the buffer is an RB (tris) buffer.

22. The kit of claim 15, wherein the first or second probe comprises one or more of the following sequences:
SEQ ID NO. 1:
5′ GTATGGTTGGTAACCAACA/IFAMdCN/CATT 3′
(SARS-CoV-2 WT probe S gene for distinction
of A23063T N501Y point mutation),
SEQ ID NO. 2:
5′ AAAGGAAAGTAACAATTAAAAC/16-TAMN-dC/TTC 3′
(SARS-CoV-2 WT probe S gene for distinction
of E484K point mutation),
and
SEQ ID NO. 3:
5′ AAAGGAAAGTAACAATTAAAACCTTT 3′
(SARS-CoV-2 mutant blank oligo for
distinction of E484K point mutation).

23. The kit of claim 15, wherein the kit is configured to allow detection of the presence of the target wild-type and/or mutant nucleic acid sequence:
i) during loop-mediated isothermal amplification, and/or
ii) without any post-amplification manipulation.

24. The kit of claim 15, wherein the target nucleic acid is from a virus, optionally where in the virus SARS-CoV-2 or a variant thereof.

25. The kit of claim 15, wherein the loop-mediated isothermal amplification uses FIP, BIP, F3 and B3 primers, optionally wherein the amplification also uses loop F and/or Loop B primers.

26. The kit of claim 15, wherein a blank oligo is provided at a concentration that is equal to or in excess of the concentration of the first oligonucleotide probe.

27. The kit of claim 26, wherein the blank oligo is provided at a concentration of two or more times the concentration of the first oligonucleotide probe, optionally wherein the blank oligo is comprised at a concentration of four or more times the concentration of the first oligonucleotide probe.


SEQUENCE LISTING (45 sequences)

SEQ ID NO: 1 - DNA, 24 bp, synthetic construct
  source 1..24: mol_type = other DNA
  modified_base 20: mod_base = OTHER; note = attached FAM fluorophore
gtatggttggtaaccaacaccatt

SEQ ID NO: 2 - DNA, 26 bp, synthetic construct
  source 1..26: mol_type = other DNA
  modified_base 23: mod_base = OTHER; note = attached TAMRA fluorophore
aaaggaaagtaacaattaaaaccttc

SEQ ID NO: 3 - DNA, 26 bp, synthetic construct
  source 1..26: mol_type = other DNA
aaaggaaagtaacaattaaaaccttt

SEQ ID NO: 4 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgctattcagggattgactgataccggaagacatctcaaatgaagtggtctatgcgacagagac

SEQ ID NO: 5 - DNA, 21 bp, synthetic construct
  source 1..21: mol_type = other DNA
tgtacgctattcagggattga

SEQ ID NO: 6 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgctattcagggattgcctgataccggaagacatctcaaatgaagtggtctatgcgacagagac

SEQ ID NO: 7 - DNA, 22 bp, synthetic construct
  source 1..22: mol_type = other DNA
tgtacgctattccagggattgc

SEQ ID NO: 8 - DNA, 19 bp, synthetic construct
  source 1..19: mol_type = other DNA
gagaccactgaccgatcta

SEQ ID NO: 9 - DNA, 19 bp, synthetic construct
  source 1..19: mol_type = other DNA
gaagtggtctatgcgacag

SEQ ID NO: 10 - DNA, 20 bp, synthetic construct
  source 1..20: mol_type = other DNA
cggagagataactacggtgc

SEQ ID NO: 11 - DNA, 22 bp, synthetic construct
  source 1..22: mol_type = other DNA
tgtacgctattcagggattgac

SEQ ID NO: 12 - DNA, 38 bp, synthetic construct
  source 1..38: mol_type = other DNA
gacagaggggctgtaagcggaccatctgtgtggtactg

SEQ ID NO: 13 - DNA, 41 bp, synthetic construct
  source 1..41: mol_type = other DNA
cgctgacgtctgtagtctagctgagatgtcttccggtatca

SEQ ID NO: 14 - DNA, 200 bp, synthetic construct; identical to SEQ ID NO: 4
  source 1..200: mol_type = other DNA

SEQ ID NO: 15 - DNA, 21 bp, synthetic construct
  source 1..21: mol_type = other DNA
  modified_base 12: mod_base = OTHER; note = attached TAMRA fluorophore
tgtacgctattcagggattga

SEQ ID NO: 16 - DNA, 200 bp, synthetic construct; identical to SEQ ID NO: 6
  source 1..200: mol_type = other DNA

SEQ ID NO: 17 - DNA, 21 bp, synthetic construct
  source 1..21: mol_type = other DNA
tgtacgctattcagggattgc

SEQ ID NO: 18 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgctattcagggatttactgataccggaagacatctcaaatgaagtggtctatgcgacagagac

SEQ ID NO: 19 - DNA, 21 bp, synthetic construct
  source 1..21: mol_type = other DNA
tgtacgctattcagggattta

SEQ ID NO: 20 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgctattcagggatttcctgataccggaagacatctcaaatgaagtggtctatgcgacagagac

SEQ ID NO: 21 - DNA, 21 bp, synthetic construct
  source 1..21: mol_type = other DNA
tgtacgctattcagggatttc

SEQ ID NO: 22 - DNA, 200 bp, synthetic construct; identical to SEQ ID NO: 4
  source 1..200: mol_type = other DNA

SEQ ID NO: 23 - DNA, 21 bp, synthetic construct
  source 1..21: mol_type = other DNA
  modified_base 12: mod_base = OTHER; note = attached TAMRA fluorophore
tgtacgctattcagggattga

SEQ ID NO: 24 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgctatttagggattgactgataccggaagacatctcaaatgaagtggtctatgcgacagagac

SEQ ID NO: 25 - DNA, 21 bp, synthetic construct
  source 1..21: mol_type = other DNA
tgtacgctatttagggattga

SEQ ID NO: 26 - DNA, 200 bp, synthetic construct
  source 1..200: mol_type = other DNA
agcccacggagaccactgaccgatctacctgaacggcgaccatctgtgtggtactggggcggagagataactacggtgccgcttacagcccctctgtcgtcgctgacgtctgtagtctagcctcattatgattgtacgcttttcagggattgactgataccggaagacatctcaaatgaagtggtctatgcgacagagac

SEQ ID NO: 27 - DNA, 21 bp, synthetic construct
  source 1..21: mol_type = other DNA
tgtacgcttttcagggattga

SEQ ID NO: 28 - RNA, 7680 nt, SARS-CoV-2
  source 1..7680: mol_type = genomic RNA
cacattacattaaactgaggaaactcgtgaccgagtctcagcagaagtagtttaaacgtcgtcctaggtgttcttgttgtcgggaactctgttgatgtcgttgaccagtatgtcgtttcgtattaacagtggtaatgataccgttagttcggtcgatattttggatcggtttacatggtaccggtaaaatatatgacgagtatgaaaggttcaagaacctctagctactctctaagtaaatttaagaaccgttggagtaactccgccagttaaagaaaaacttacaaatgttgacttcgtaattacggtctctacagtggatttagttgtagaccactacatactaagaattttataaatagattgaggaggaacttactcagattaagtccaacgtttcctagtatttgacacaacaactgttaaggataatgttgtagtgtcaatggtctgtgtttacacaacagacatcattactaaacaccaagtatttttaaggaaacacaatgtttggtcacacacggtaaactttgtttctgtggaagtgctcctttcacacgaaaaggtagtactgtttaccgtcctcgtcaacacttcaagaaaagaacacgtccctgtattcagtgtacgttcttctgatgtggtactccacgactgactcccttcctgtattctactatcgggaaaggtgtttttagttgagaaaaactaacaggttcatgtgtgagactgtaaaatcatcgtcgttctaatcgtcttcgagactaaagacgtcgagattaattaacaactcagtgtatacagacgtttgaaacttcagacggacactagttggatagttaaacgtgaagtcggagttgaaacagttctgcactttcctatagtaaattttgtgaactttaacgtggttttaacctcgattcaacaaattgttcgcacaaatttcgaacacgtaaaaccaactggtgtagaacttcaaaaggttcacgtgaacgacaccttctttcactcagaacttaaaacggttatcgtgataatttaaccaaccgttagttaaaaaccaagagtatctcttgtaagacacattgaggttatggtaatttggatattcggtaaacgtatcgtttaccataaacattacgtcgtggacgtggtttccaggttggtcttcactaacatgggcgattgtcacgtcttcacataactcgttagtaaagtagacactcgtttccaccgttttgtcattccggcaatttgaaaacacgtgtttactccagagatcgtcgttatagtggttccgttagtggtataacaaactacttcggtcgtagacgttcacagtgaaacaacttttcatctagaagttatttactggagaacgaaccaaaactacctagaccattataaacactttttaattttggtggttttagaaattaaccaccacaaaacatttaaacaaactgaacacgtttttgaagaacccacaaaaacagaacaagttgtcgataaggtcaatttcgtgccaaattaacacatgtttttgacggtataacgttgttttctaacgacgtaagtcaacttagtggtgtttacatgtaacatgttagatgactacagaaccagtatctgtgaccatcttaaagacaccattgtgattatcattttaaacacccataccgttatctcaataatctcattcgttgacttaaaagacgtggttcactgtatcacatccgttactacctaactgatcgatgtgatgcacgggcggctcctcttaatcagactcagactattgatcgcgtatatggacgtggttacccatacagtgtgagtatactcaacaactgtacaagtcggggataatttgtcggacgtgcacaaactttttgtaatcttggacatcttatttgtgcggttcatcctcattcaactagacgtacttatcgttgtccctgaagacacgtcaattgtaggactatttcttgtcgttggaccaatcttcataaacaaggaccacaatattgtgactgtggtggttttcttgtaccacattacagttcttagagttcacagacacctagtgcctgtcgtagtcatcacagtcgttacagagacggtttaacaacctttccgtctttgaaaaacaatctgagtcattcttgtggacacggacaatttggtaacttcaactttaactgtgtaaacaaaaattggtttaatcatctgaaaaatccaggtgtttgtcaacgaccacgtacatcttcaagttttctttcatgatgatgagacataccaaccattggttgtggtaatcacccaacctttggtatactaacatttcctttcattgttaattttggaagttgtggtaatgttccacacgatggccggactatctaaagtcaactttatagagagagttttccaaactctaatctgaaggatttgttagatatgtccattaatattaatggtggttggaatcttagttctaacaatcttaaggttcgatattgcgtcggacattttagtagaccattaaatattaatattagtcgttagaaaggtcaaacgggacctcgctaaacagactgaagtagtggagattaatgtttacttagacgtatctgtaatcatttcgtctctagtaaattaaatcatcctctgtgaggtattgtgaattttcaccttttactacgccttaatatatcctgtcttattagtcgttgtgtcaacgactaagagaaggacaaggttcgtatttgtctacgtttagaccaccgcaatttttgaagtggttttcccgtgttcaaacattataatcctttagattgttatctaagacaaccaacctgagatttcaatcttcaaactatctaaggaaaaagatgtcacttcctaaagttgcatgtgaaacaaagactctctcccagttcacgtgtcagatgtcgtagacattaccaaggtaaaagtaatataaaattatcttttcaggatccaacttctattgggtgtattattcgacgtcgtggtcgacaggttggacttcttcttagtggtcctcagtttattgaagatacatttcgttcatttcaaactttggatcactacaattatggataaccgtttagatggttaccaagatttcggctttttgggactccctctagtgcgtgatttaattatccgcacacgaatcttatataaaattttattggtagttataagaatttgtgtttaagggattctaaaaactttaatgggacaaaaggaagttccaggtattcttttccgactctctgtataagttttcacgttaataagcgtgatcttatttgagacttgagtgaaaggtaggttgaaaacaacaaaaacaccattatttgtgggtttttacctagtaatgttttaactttaagtgtctgaaattattgttgtaatcatcgcaataattgttattcatccctgacccagaagcttagatttcatcatggtttttaggtcggagaataatacaatctgaagagtcaccttcgttttatttgtggtagtaatttaccatcctgtcccaatagtttggagaatcatggtaaccagggtctctgtacatatcgtaccttggttcattgtaaccttttctttccattcttgttcaggactcaacttacattttgactcctagacttttgaaacagtcccattatttgtggtgcacactttcttaatcacatacgtcccccattaactcaagaccaacattctaattgtgtgactgatctctgatcaccgttattttgttctttttgtttgtaacaagcaaatcaacaattgttccacattacattaaactgaggaaactcgtgaccgagtctcagcagaagtagtttaaacgtcgtcctaggtgttcttgttgtcgggaactctgttgatgtcgttgaccagtatgtcgtttcgtattaacagtggtaatgataccgttagttcggtcgatattttggatcggtttacatggtaccggtaaaatatatgacgagtatgaaaggttcaagaacctctagctactctctaagtaaatttaagaaccgttggagtaactccgccagttaaagaaaaacttacaaatgttgacttcgtaattacggtctctacagtggatttagttgtagaccactacatactaagaattttataaatagattgaggaggaacttactcagattaagtccaacgtttcctagtatttgacacaacaactgttaaggataatgttgtagtgtcaatggtctgtgtttacacaacagacatcattactaaacaccaagtatttttaaggaaacacaatgtttggtcacacacggtaaactttgtttctgtggaagtgctcctttcacacgaaaaggtagtactgtttaccgtcctcgtcaacacttcaagaaaagaacacgtccctgtattcagtgtacgttcttctgatgtggtactccacgactgactcccttcctgtattctactatcgggaaaggtgtttttagttgagaaaaactaacaggttcatgtgtgagactgtaaaatcatcgtcgttctaatcgtcttcgagactaaagacgtcgagattaattaacaactcagtgtatacagacgtttgaaacttcagacggacactagttggatagttaaacgtgaagtcggagttgaaacagttctgcactttcctatagtaaattttgtgaactttaacgtggttttaacctcgattcaacaaattgttcgcacaaatttcgaacacgtaaaaccaactggtgtagaacttcaaaaggttcacgtgaacgacaccttctttcactcagaacttaaaacggttatcgtgataatttaaccaaccgttagttaaaaaccaagagtatctcttgtaagacacattgaggttatggtaatttggatattcggtaaacgtatcgtttaccataaacattacgtcgtggacgtggtttccaggttggtcttcactaacatgggcgattgtcacgtcttcacataactcgttagtaaagtagacactcgtttccaccgttttgtcattccggcaatttgaaaacacgtgtttactccagagatcgtcgttatagtggttccgttagtggtataacaaactacttcggtcgtagacgttcacagtgaaacaacttttcatctagaagttatttactggagaacgaaccaaaactacctagaccattataaacactttttaattttggtggttttagaaattaaccaccacaaaacatttaaacaaactgaacacgtttttgaagaacccacaaaaacagaacaagttgtcgataaggtcaatttcgtgccaaattaacacatgtttttgacggtataacgttgttttctaacgacgtaagtcaacttagtggtgtttacatgtaacatgttagatgactacagaaccagtatctgtgaccatcttaaagacaccattgtgattatcattttaaacacccataccgttatctcaataatctcattcgttgacttaaaagacgtggttcactgtatcacatccgttactacctaactgatcgatgtgatgcacgggcggctcctcttaatcagactcagactattgatcgcgtatatggacgtggttacccatacagtgtgagtatactcaacaactgtacaagtcggggataatttgtcggacgtgcacaaactttttgtaatcttggacatcttatttgtgcggttcatcctcattcaactagacgtacttatcgttgtccctgaagacacgtcaattgtaggactatttcttgtcgttggaccaatcttcataaacaaggaccacaatattgtgactgtggtggttttcttgtaccacattacagttcttagagttcacagacacctagtgcctgtcgtagtcatcacagtcgttacagagacggtttaacaacctttccgtctttgaaaaacaatctgagtcattcttgtggacacggacaatttggtaacttcaactttaactgtgtaaacaaaaattggtttaatcatctgaaaaatccaggtgtttgtcaacgaccacgtacatcttcaagttttctttcatgatgatgagacataccaaccattggttgtggtaatcacccaacctttggtatactaacatttcctttcattgttaattttggaagttgtggtaatgttccacacgatggccggactatctaaagtcaactttatagagagagttttccaaactctaatctgaaggatttgttagatatgtccattaatattaatggtggttggaatcttagttctaacaatcttaaggttcgatattgcgtcggacattttagtagaccattaaatattaatattagtcgttagaaaggtcaaacgggacctcgctaaacagactgaagtagtggagattaatgtttacttagacgtatctgtaatcatttcgtctctagtaaattaaatcatcctctgtgaggtattgtgaattttcaccttttactacgccttaatatatcctgtcttattagtcgttgtgtcaacgactaagagaaggacaaggttcgtatttgtctacgtttagaccaccgcaatttttgaagtggttttcccgtgttcaaacattataatcctttagattgttatctaagacaaccaacctgagatttcaatcttcaaactatctaaggaaaaagatgtcacttcctaaagttgcatgtgaaacaaagactctctcccagttcacgtgtcagatgtcgtagacattaccaaggtaaaagtaatataaaattatcttttcaggatccaacttctattgggtgtattattcgacgtcgtggtcgacaggttggacttcttcttagtggtcctcagtttattgaagatacatttcgttcatttcaaactttggatcactacaattatggataaccgtttagatggttaccaagatttcggctttttgggactccctctagtgcgtgatttaattatccgcacacgaatcttatataaaattttattggtagttataagaatttgtgtttaagggattctaaaaactttaatgggacaaaaggaagttccaggtattcttttccgactctctgtataagttttcacgttaataagcgtgatcttatttgagacttgagtgaaaggtaggttgaaaacaacaaaaacaccattatttgtgggtttttacctagtaatgttttaactttaagtgtctgaaattattgttgtaatcatcgcaataattgttattcatccctgacccagaagcttagatttcatcatggtttttaggtcggagaataatacaatctgaagagtcaccttcgttttatttgtggtagtaatttaccatcctgtcccaatagtttggagaatcatggtaaccagggtctctgtacatatcgtaccttggttcattgtaaccttttctttccattcttgttcaggactcaacttacattttgactcctagacttttgaaacagtcccattatttgtggtgcacactttcttaatcacatacgtcccccattaactcaagaccaacattctaattgtgtgactgatctctgatcaccgttattttgttctttttgtttgtaacaagcaaatcaacaattgttc

SEQ ID NO: 29 - RNA, 24 nt, SARS-CoV-2
  source 1..24: mol_type = genomic RNA
aatggtgttggttaccaaccatac

SEQ ID NO: 30 - RNA, 24 nt, SARS-CoV-2
  source 1..24: mol_type = genomic RNA
gtatggttggtaaccaacaccatt

SEQ ID NO: 31 - DNA, 24 bp, synthetic construct
  source 1..24: mol_type = other DNA
  modified_base 20: mod_base = OTHER; note = attached FAM fluorophore
aatggtgttggttaccaaccatac

SEQ ID NO: 32 - DNA, 22 bp, synthetic construct
  source 1..22: mol_type = other DNA
  modified_base 15: mod_base = OTHER; note = attached TAMRA fluorophore
tgtttttaacaaagcttggcgt

SEQ ID NO: 33 - DNA, 24 bp, synthetic construct
  source 1..24: mol_type = other DNA
ctgtatagattgtttaggaagtct

SEQ ID NO: 34 - DNA, 18 bp, synthetic construct
  source 1..18: mol_type = other DNA
gctggtgcatgtagaagt

SEQ ID NO: 35 - DNA, 46 bp, synthetic construct
  source 1..46: mol_type = other DNA
tcaacaccattacaaggtgtgctaaatctcaaaccttttgagagag

SEQ ID NO: 36 - DNA, 43 bp, synthetic construct
  source 1..43: mol_type = other DNA
tgctggtattgttggtgtactgggtttgtatgaaatcaccgaa

SEQ ID NO: 37 - DNA, 24 bp, synthetic construct
  source 1..24: mol_type = other DNA
cggcctgatagatttcagttgaaa

SEQ ID NO: 38 - DNA, 21 bp, synthetic construct
  source 1..21: mol_type = other DNA
aaaacccagatatattacgcg

SEQ ID NO: 39 - DNA, 19 bp, synthetic construct
  source 1..19: mol_type = other DNA
ctacaacaggaactccact

SEQ ID NO: 40 - DNA, 39 bp, synthetic construct
  source 1..39: mol_type = other DNA
cgcatggcatcacagaattgtcgccaacttaggtgaacg

SEQ ID NO: 41 - DNA, 43 bp, synthetic construct; identical to SEQ ID NO: 36
  source 1..43: mol_type = other DNA

SEQ ID NO: 42 - DNA, 22 bp, synthetic construct
  source 1..22: mol_type = other DNA
tgtttttaacaaagcttggcgt

SEQ ID NO: 43 - DNA, 23 bp, synthetic construct
  source 1..23: mol_type = other DNA
tcaagatctcaatggtaactggt

SEQ ID NO: 44 - DNA, 21 bp, synthetic construct
  source 1..21: mol_type = other DNA
  modified_base 12: mod_base = OTHER; note = attached TAMRA fluorophore
tgtacgctattcagggattga

SEQ ID NO: 45 - DNA, 22 bp, synthetic construct
  source 1..22: mol_type = other DNA
  modified_base 12: mod_base = OTHER; note = attached Cy5 fluorophore
tgtacgctattccagggattgc